(19) United States
(12) Patent Application Publication
(10) Pub. No.: US 2008/0171384 A1
(43) Pub. Date: Jul. 17, 2008

(54) Method of obtaining a population of human haemopoietic stem cells

(75) Inventors: Elaine Dzierzak, Rotterdam (NL); Katrin Ottersbach, Rotterdam (NL); Catherine Robin, Rotterdam (NL); Sandra C. Mendes, Gouda (NL)

Correspondence Address: EDWARDS ANGELL PALMER & DODGE LLP, P.O. BOX 55874, BOSTON, MA, 02205, US

(73) Assignee: Erasmus University Medical Center

(21) Appl. No.: 11/895,351

(22) Filed: Aug. 24, 2007

Related U.S. Application Data
(63) Continuation of application No. PCT/IB2006/000745, filed on Feb. 27, 2007.

(30) Foreign Application Priority Data
Feb. 25, 2006 (GB) 0503918.5

(51) Int. Cl. C12N 5/08

(52) U.S. Cl. 435/366

(57) ABSTRACT

The present invention relates to a method for the isolation of large numbers of hematopoietic stem cells. In particular the invention relates to a method for the isolation of viable haemopoietic stem cells from the placenta. The uses of these isolated cells in various applications are also described.

### RELATED APPLICATIONS

This is a continuation patent application that claims priority to PCT patent application number PCT/IB2006/000745, filed on Feb. 27, 2007, which claims priority to GB patent application number 0503918.5, filed on Feb. 25, 2005, the entirety of which are herein incorporated by reference.

### FIELD OF INVENTION

The present invention relates to a method for the isolation of large numbers of haemopoietic stem cells. In particular the invention relates to a method for the isolation of viable haemopoietic stem cells from the placenta. The placenta which is usually discarded represents a rich source of potent transplantable hematopoietic stem cells. The uses of these isolated cells for clinical transplantation therapies are also described.

### BACKGROUND

The haernopoietic system is a complex hierarchy of cells of different mature cell lineages. These include, amongst others, the cells of the immune system that offer protection from invading pathogens, the cells that carry oxygen through the body and cells involved in wound healing. All these mature cells are derived from a pool of haemopoietic stem cells (HSCs) that, in the adult, reside in the bone marrow and that are capable of self-renewal and differentiation into any blood lineage. It is these cells that are also often targets of mutations that result in a number of blood-related diseases and/or malignancies. It is therefore not surprising that these cells have been the subject of intense studies due to their enormous potential in clinical applications. Studies have focused on ways of harvesting HSCs from different sources, on ways of enriching for them, expanding their limited numbers outside the body, storing them and manipulating them. As these cells have the ability to replenish the entire haemopoietic system, they are not only currently used for transplantations following haemotoxic insults such as radiotherapy or chemotherapy or for the replacement of leukaemic cells, but they are also attractive targets for gene therapy, since the genetic manipulation done in HSCs will be passed on to all the blood lineages as these cells differentiate. There have also been a number of reports in the last few years that claim that HSCs are not only restricted to producing haernopoietic progeny, but may also be able to differentiate into cell of the liver, muscle, gut and brain in a process termed plasticity. If these claims can be substantiated, it would open up a vast array of new HSC-based therapies for diseases such as muscle-wasting diseases or neurodegenerative diseases (reviewed in (de Vries et al., 2004; Pamphilon, 2004; Peterson, 2004)).

Conventionally, HSCs have been obtained from the bone marrow of donors (autologous or allogeneic). Despite the fact that relatively high numbers of HSCs can be obtained in this way, it is a rather cumbersome and invasive process that requires general anesthetics for the donor and further handling and purification processes (Parfphilon, 2004). Other possible sources of HSCs have therefore been explored. Apart from residing in the bone marrow, HSCs have also been observed to circulate in the peripheral blood. The number of these circulating HSCs can be increased either by insults to the haemopoietic system or by administering mobilising factors such as G-CSF. This source of HSCs is much more accessible than HSCs from the bone marrow, yet it also requires pretreatment of the donor and substantial amounts of blood being taken from the donor, especially since the yield is lower (de Vries et al., 2004; Pamphilon, 2004).

A third source of HSCs has received increasing attention in recent years (reviewed in (Rocha et al., 2004). It was found that the umbilical cord vessels of newborns contain HSCs (Benito et al., 2004; Cohen and Nagler, 2004; de Vries et al., 2004; Pamphilon, 2004). This tissue is normally discarded and therefore obtaining this tissue requires no invasive procedures for the donor. It was also observed that cord blood (CB) HSCs are more naive than adult HSCs which results in less stringent criteria for ILA matching (allowing a mismatch in 1-2 loci) which makes these cells available for unrelated, partially matched recipients and thus to a much wider population (Benito et al., 2004; Cohen and Nagler, 2004; de Vries et al., 2004). The consequence of using a mismatched graft is a complication known as graft-versus-host-disease (GVHD), which not only results in graft rejection, but can also have a serious health impact. By using these more naive CB HSCs, it was observed that the incidence of GVHD can be reduced. Other advantages of CB HSCs include their higher proliferative potential, a lower transmission rate of infectious and genetic diseases, an immediate availability of the product, thus avoiding the risks for the donor and loss of registries, and the relative ease with which HSCs can be obtained from the CB (Benito et al., 2004; de Vries et al., 2004). Protocols have also been established that allow the freezing and thus long-term storage (more than 15 years) of CB HSCs which has resulted in the establishment of a number of CB banlks all over the world (Benito et al., 2004; de Vries et al., 2004; Pamphilon, 2004). These cells retain their stem cell potential during the storage period. This not only greatly increases the chance of finding matched grafts, but also allows the storage of an individuals own HSCs for a long period of time, thus making them available at later times in that individuals life if required. Although over 85,000 cryopreserved CB units are ready for clinical use, the major disadvantage of CB HSCs is their low number per CB unit, which is 10 times less than what can be obtained from 1 bone marrow unit (Benito et al., 2004; Cohen and Nagler, 2004; de Vries et al., 2004). This can result in a higher risk of graft failure and a delayed haemopoietic recovery. For this reason, CB grafts have primarily been used for paediatric patients. As inventoried recently by Netcord (Rocha et al., 2004), the cooperative network of experienced CB banks, 1815 children and 982 adults have been transplanted with CB cells.

Like the cord blood, the placenta is derived from fetal cells and contains hematopoietic cells. Most work concerning the haemopoietic potential of the placenta has been performed in the mouse model. The chorioallantoic placenta forms at the junction of the allantois and chorion and thereafter, the allantois contributes the fetal vascular and associated stromal components, including the umbilical vessels (Downs et al., 1998). In the chick embryo, the allantois has been identified as a haemopoietic site (Caprioli et al., 1998). However, in murine embryos it is not yet clear whether the allantois is haemopoietic (Downs and Harmann, 1997). The murine allantois derives from the mesoderm at the posterior end of the mouse embryo and makes contact with the chorion at E8.5 (reviewed in (Cross, 1998; Cross et al., 2003a; Cross et al., 2003b; Han and Carter, 2001; Rossant and Cross, 2001). It is at this time that the mouse placenta contains its first haemopoietic activity in the form of clonogenic haemopoietic progenitors including CFU-GMs, CFU-GEMMs, BFU-Es and HPP-CFCs (Alvarez-Silva et al., 2003). Moreover, between E10 and E12 these progenitors were present at numbers higher than those found in the other sites of fetal haemopoietic generation, the yolk sac, aorta-gonad-mesonephios (AGM) and fetal liver. Progenitor numbers increase until E17 just before birth. However, in these studies it was not investigated whether the placenta also contains HSCs.

In a recent abstract of the American Society for Hematology meeting (Mikkola et al., 2004), it was reported that the mouse placenta contains HSCs. The onset of HSC activity in the placenta occurs at E10.5/11.0 and expands until E12.5/13.5. Expansion of placental HSCs is 17-fold as compared to the 2-fold expansion of clonogenic progenitors at E11.5/12.5. However, it was found by the authors that HSC cell activity declines towards the end of gestation. Thus making the mouse placenta an unsuitable source of HSCs.

Thus, there remains in the art a need for an abundant source of HSCs for therapeutic and other uses.

### SUMMARY OF THE INVENTION

The present inventors have surprisingly found, contrary to previous suggestions concerning mouse placenta, that the human placenta at the time of birth of an individual is a rich source of HSCs. In addition, the inventors have found that HSCs isolated from the placenta are of a more naive nature with a higher proliferative capacity than HSCs isolated from peripheral blood or bone marrow.

Thus in a first aspect, the present invention provides a method for obtaining a population of haemopoietic stem-cells (HSCs) which method comprises extracting those cells from a placenta of a human individual post-partum.

Isolation of HSCs from the placenta follows a well established protocol and will be familiar to those skilled in the art, and despite requiring more handling steps than cord blood (CB) HSC isolation, results in higher numbers of HSCs. As with cord blood HSCs, routine record keeping for HLA type, storage and a network of banks can easily be established, thus resulting in the immediate availability of low risk HSC grafts.

Thus, in a further aspect the present invention provides a population of haemopoietic stem cells (HSC) isolated from the placenta of a human individual postpartum.

According to the above aspect of the invention, HSCs isolated from the human placenta post-partum are of a more naive nature and/or have higher proliferative capacity than HSCs isolated from peripheral blood or bone marrow.

Placental HSCs according to the invention are suitable for storage by freezing methods that follow a well established protocol and will be familiar to those skilled in the art. As with cord blood, human placental HSCs maintain the viability and function after storage in liquid nitrogen.

Thus, in a further aspect the present invention provides a population of haemopoietic stem cells isolated from the placenta of a human individual post-partum that maintain their viability and function after storage.

The present inventors have found that populations of HSCs isolated from human placenta may be expanded (that is increased in number) by treating those cells ex vivo with any one or more of the growth factors in the group consisting of the following: IL-3, IL-6, Tpo, OSM, SCF, GM-CSF, MIP1γ, Wnt, BMP, NGFβ. This list is non limiting. Further, those skilled in the art will be aware of other growth factors suitable for the expansion of HSC's.

Therefore, in a further aspect the present invention provides a method for providing a population of HSCs comprising the steps of:
(a) Extracting a population of HSCs from human post-partum placenta, and
(b) Treating that population of cells with one or more growth factors in the group consisting of: IL-3, IL-6, Tpo, OSM, SCF, GM-CSF, MIP1γ, Wnt, BMP, NGFβ;
(c) Optionally treating them with explant cultures derived from placenta, and/or with explant cultures derived from aorta gonad mesonethros (AGM) and/or stromal cells lines derived from these tissues.

These treatments (b) and (c) referred to above can be performed individually (that is either one or the other treatment) or in combination. Further, when administered in combination, the treatments may be administered either sequentially or simultaneously.

In yet a further aspect the invention provides a population of HSCs extracted from human placenta according to the present invention, wherein the number of HSCs within that population has been increased by treatment of that cell population with any one or more growth factors in the group consisting of the following: IL-3, IL-6, Tpo, OSM, SCF, GM-CSF, MIP1γ, Wnt, BMP, NGFβ Further, human HSC's according to the invention may be treated with explant cultures derived from placenta, and/or with explant cultures derived from aorta gonad mesonethros (AGM) and/or stromal cells lines derived from these tissues. These treatments (treatment with growth factors and explant cultures/stromal cells may be performed individually (that is either one or the other treatment) or in combination. Further, when administered in combination, the treatments may be administered either sequentially or simultaneously.

In yet a further aspect, the present invention provides the use of HSCs isolated from the human placenta of an individual postpartum, in therapy.

In yet a further aspect the present invention provides the use of HSCs isolated from the human placenta of an individual post-partum in populating an individual with haemopoietic stem cells.

In yet a further aspect the present invention provides the use of HSCs isolated from the placenta of a human individual post-partum in populating a variety of non-hematopoietic tissues of an individual with haemopoietic stem cells.

In yet a further aspect the present invention provides the use of HSCs isolated from the placenta of a human individual post-partum in populating a variety of non-hematopoietic tissues of an individual with ex vivo treated/manipulated haemopoietic stem cells.

### DEFINITIONS

Haemopoietic stem cells (HSCs): Pluripotent stem cells are found in certain organs of the body such as the bone marrow and cord blood. These stem cells form cells of all mature blood cell lineages and are thus capable of re-colonising the entire immune system and the erythroid and myeloid lineages in all the haemopoietic tissues such as bone marrow, spleen, thymus, etc. In addition these cells are capable of self-renewal. They provide for life-long production of all lineages of haemopoietic cells. It is these cells that are also often targets of mutations that result in a number of blood-related diseases and/or malignancies. Such haemopoietic stem cells are typically of low forward scatter and side scatter profile by flow cytometric procedures. Some are metabolically quiescent as demonstrated by Rhodamine labeling which allows determination of mitochondrial activity. Haemopoietic stem cells comprise certain cell surface markers such as CD34, CD45, c-kit, Sca-1, PLCP, Flk-1, Mac-1, CD31, VE-cadherin, endoglin, etc. They can also be defined as cells lacking the expression of the cell surface CD38 marker for example. However, expression of some of these markers is dependent upon the developmental stage and tissue specific context of the HSC. Some HSCs called “side population cells” exclude the Hoescht 33342 dye as detected by flow cytometry. Thus, HSCs have descriptive characteristics that allow for their identification and isolation.

Naïve haemopoietic stem cells: Haemopoietic stein cells from embryonic, fetal and early post-partum sources can be considered naive. Naive defines those HSCs that are the direct (or almost the direct) progeny of non-haemopoietic cells, for example the direct progeny of haemangioblasts (common precursor cells for endothelial and haemopoietic lineages) or endotlielial cells with haemopoietic potential. Naive HSCs possess the most extensive proliferative potential since they are young. They can be amplified to greater numbers of progeny than older bone marrow derived HSCs. They have a wider range of differentiation potentials and for example may generate special T and B lymphocytes that adult bone marrow HSCs cannot. Importantly naive HSCs are less likely to elicit a graft-versus-host response and therefore, can be used clinically for transplantations in which a matched donor is not available.

A population of haemopoietic stem cells: According to the present invention, a population of haemopoietic stem cells refers to more than one stem cell. Preferably, it refers to more than 2, 5, 10, 20, 50, 100, 200, 500, 1000, 2000, 5000 or 10,000 20,000 or 50,000 cells.

Populating/Repopulating an individual with haemopoietic stem cells: According to the invention described herein, the term refers to increasing the numbers and/or functional activity of haemopoietic stem cells in an individual. In a preferred embodiment of the invention, the individual is a human.

The term ‘extracting those cells’ (from the placenta of a human individual) refers to the process of substantially separating those HSCs from human placental tissue. Those skilled in the art will appreciate that as a result of the extraction process the HSC cell population obtained may not be 100% pure. For example there may be present other cell types, cell and tissue matrix material and so on. Advantageously, the extraction process will result in an HSC cell population which comprises 40%, 45%, 50%, 55%, 60%, 65%, 70%, 75%, 80%, 85%, 88%, 89%, 90%, 92%, 94%, 96%, 98%, 99% HSCs as compared with other cell types. Most advantageously, the extraction process will result in an HSC cell population which comprises 100% HSCs as compared with other cell types.

### BRIEF DESCRIPTION OF THE FIGURES

FIGS. 1 (A, B, E) describes HSCs clusters along the vasculature of a developing mouse embryo and (C, D, F-I) the development of the placenta.

FIG. 2_describes the presence of placental haemopoietic stem cells in mouse embryos at mid-gestation. A) shows high level, multi-hematopoietic lineage repopulation by placental HSCs in the hematopoietic tissues of transplanted mice. B-I) shows FACS data phenotypically indicating HSCs in the placenta.

FIG. 3 describes the distribution of haemopoietic stem cells in mouse placentas with antibodies specific for hematopoietic markers (A-D, M) CD31, (E-H, N) CD34 and (I-L, O) CD41.

FIG. 4 describes histological expression studies of hematopoietic transcription factors and demonstrates the localisation of haemopoietic stem cells within mouse placentas.

FIG. 5 The post-partum placenta contains haemopoietic stem cells.

FIG. 6 Growth factors MIP1γ and NGFβ increase and BMP antagonists (gremlin and noggin) decrease haemopoietic stem cell numbers in mouse AGM stromal cell cocultures.

FIG. 7 shows (A) the structure of the human placenta and the segments used for HSC isolation and (B) shows FACS evidence of phenotypically defined HSCs in the post-partum human placenta.

FIG. 8 shows the presence of potent single and multilineage hematopoietic progenitor (CPU; colony-forming unit) activity in post-partum human placenta.

FIG. 9 shows evidence of functional HSCs in post-partum human placenta as measured by hematopoietic repopulation in the SCID-NOD xenotransplantation assay (A) In vivo method to test for placental HSCs (B) FACS analysis of recipient blood and (C) PCR analysis of recipient blood.

FIG. 10 shows that ex vivo culture of mouse placenta tissue with IL-3 increases HSCs 1.8- to 5.0-fold.

### DETAILED DESCRIPTION OF THE INVENTION

(A) Extraction of HSCs from Human Post-Partum (Post-Birth) Placenta.

In a first aspect the present invention provides a method for obtaining a population of haemopoietic stem cells (HSCs) which method comprises extracting those cells from a placenta of a human individual post-partum.

According to the method of the invention, the term ‘extracting those cells’ (from the placenta of an individual) refers to the process of substantially separating those HSCs from placental tissue. Those skilled in the art will appreciate that as a result of the extraction process the HSC cell population obtained may not be 100% pure. For example there may be present other cell types, cell and tissue matrix material and so on. Advantageously, the extraction process will result in an HSC cell population which comprises 40%, 45%, 50%, 55%, 60%, 65%, 70%, 75%, 80%, 85%, 88%, 89%, 90%, 92%, 94%, 96%, 98%, 99% HSCs as compared with other cell types. Most advantageously, the extraction process will result in an HSC cell population which comprises 100% HSCs as compared with other cell types.

### Extraction Techniques.

### (i) FACs Sorting.

FACS (fluorescence activated cell sorting) analysis may be used to sort and separate HSCs in the placenta from other cells types on the basis of one or more cell surface markers which are found on the surface of HSCs and are advantageously absent on the surface of other cell types present in placental tissue or vica versa.

In a preferred embodiment of the above aspect of the invention the cell surface marker is any one or more of those in the group consisting of the following: CD34, CD38, CD31, c-kit, Flk-1, KDR, Mac-1, CD45, Sca1, Hoescht 33342 exclusion, endoglin, PLCP (Robin et al., 2004)

The FACS allows cells to be separated and isolated through the use of tagged antibodies, anti-immunoglobulins, or other binding proteins or moieties. The sample mixture is funnelled through a nozzle which isolates cells and charges them. At the base of the FACS, there are 2 variably charged deflection plates and cell collectors. In order to carry out FACS, the individual cells with similar size and density to the resting lymphocyte pool must be isolated. This is accomplished by suspending the cells in a fluid (e.g., saline). This suspension of cells is then forced through a fine, high-pressure nozzle or fluidic diluting system which distributes the cells into a single-file line or flow cell (Kidd and Nicholson 2292). Light is an integral part of the FACS technique. Light striking each cell is scattered. By using electronic devices that measure scattered light and fluorescence, different types of cells and their sizes can be identified. FACs have two types of data collecting hardware: light scatter sensors and photomultiplier tubes (PMTs). The light scatter sensors measure the light that is scattered by each cell from two different angles. The forward angle light scatter sensor (FALS) gathers light scattered in the forward direction. This type of scattered light gives a clue as to a cell's size. Right angle, orthogonal, or side light scatter (SS) sensors gather light scattered at 90° from the original direction of the light source. This light reveals cell granularity, refractiveness, and the presence of intracellular structures that reflect light (Darzynkiewicz, et al. 335). Scatter sensors are useful in distinguishing cells based on the cells' different structures. Neutrophils, for example, display more SS than lymphocytes (Kidd and Nicholson 229). In addition, different cell lineages or cells at different stages of development i.e., pre B cells versus plasma B cells) can be distinguished based on their forward and side scatters (Kantor, Merrill, and Hillson 13.1). Finally, PMTs detect fluorescent emissions from the fluorescent dyes on antibodies bound to cells or from auto-fluorescence of the cells. To collect selected cells, the cells are passed through an electric field generated by oppositely-charged plates. By changing the direction of the electric field between the plates, selected cells can be directed into precise collection areas. (Apoptosis by Flow Cytometry.” Manual of Clinical Laboratory Immunology, 5th edn. Ed. Noel R. Rose, Everly Conway deMacario, James D. Folds, H. Clifford Lane, Robert M. Nalcamura. Washington, D.C.: American).

### (ii) MACs Analysis.

The process of magnetic cell sorting will be familiar to those skilled in the art. Such a technique may either in addition or as an alternative to FACs sorting in extracting a population of HSCs from post-partum placenta.

(iii) Other Methods for the Extraction of HSCs from Post-Partum Placenta.

Other methods suitable for the extraction of HSCs from human postpartum placenta include any one or more of the techniques selected from the group consisting of: adhesion to plastic, protein coated plates or matrices, density gradient fractionations.

(B) Haemopoietic Stem Cells Extracted from Post-Partum Placenta.

The present inventors have found potent HSCs in the mouse placenta beginning at E11 and increasing in number thereafter. The inventors took advantage of a well-known HSC marker (Sca1) to sort for placental HSCs and found that placental HSCs have many of the phenotypic properties of bone marrow HSCs. Most importantly, they possess potent repopulating activity when transplanted into irradiated adult recipient mice, giving rise to long-term, high level, multilineage haemopoietic repopulation.

However, unlike the Mikkola et al. study (Mikkola et al., 2004), the present inventors find HSCs in the late gestation mouse placenta. More importantly, the present inventors find abundant HSCs in the post-partum human placenta. These cells are as potent as human cord blood haemopoietic stem cells. Thus the postpartum (post-birth) human placenta is a rich source of HSCs that may be exploited for purposes of clinical transplantation. Moreover, they represent an important source of HSCs that may be stored along with cord blood for future clinical therapeutic use.

A well-recognized feature shared by tissues within the embryo that are haemopoietic is the associated development of the vascular systems. A wide range of evidence has supported hemangioblasts (a common mesodermal precursor for both the endothelial and haemopoietic lineages) and hemogenic endothelium as the presumptive precursors to emerging haemopoietic cells (reviewed in (Dieterlen-Lievre, 1998; Nishikawa, 2001). The placenta is indeed a well-vascularized tissue and thus, it is possible that both the endothelial and haemopoietic lineages are generated in parallel and perhaps from a common precursor. If this is the case, then as the placenta grows throughout development, vasculogenesis and haemopoietic stem cell emergence will also and HSC activity should be isolated in both cell lineages.

Previously the present inventors have generated Ly-6A(Sca-1)GFP transgenic mice expressing the green fluorescent protein (GFP) under the regulatory elements of the HSC marker Sca-1. In these studies it was demonstrated that all HSC activity was confined to the GFP⁺ fraction in AGM, fetal liver and adult bone marrow tissues (de Bruijn et al., 2002; Ma et al., 2002). It was further shown by immunohistologic analysis of E9-11 embryos that GFP expression localizes to the endothelial layer of the dorsal aorta, the vitelline artery and the major blood vessels of the YS. The common expression of this transgene, as well as other markers, in endothelial cells and AGM HSCs highlights the close relationship between these two cell types and supports the notion that HSCs derive from specialized ‘hemogenic’ endothelial cells that are present for at least a period of time during midgestation and perhaps at later gestational stages and early post-partum.

In extending the analysis of these HSC marker transgenic embryos to earlier times in development, E6-E7.5, high GFP expression was found restricted to extraembryonic tissues; the ectoplacental cone and the extraembryonic ectoderm. Since these sites contribute to the placenta, the temporal expression was examined in the placenta, beginning from E9, just after the fusion of the chorion and the allantois. At all stages examined, the inventors found high level GFP expression restricted to the embryonic/fetal vessels of the placental labyrinth (the layer of the placenta where the embryonic circulation comes into contact with the maternal circulation). This observation prompted them to test for HSC activity in the placenta. The inventors found that adult-type HSCs are indeed present in the placenta, starting from E11, and that they are again exclusively found in the Ly-6A(Sca-1)GFP⁺ fraction. Moreover, other HSC markers co-localize with GFP⁺ cells in the labyrinth region. These results implicate the placenta as a potent generating source of haemopoietic stem cells and that these cells may arise from the placental vascular endothelium. Moreover, these HSCs are not maternally-derived. They are derived from the embryo and thus, these cells possess high proliferative potential, extensive regenerative potential and are useful for transplantation to unrelated recipients.

Haemopoietic stem cell development in the human embryo closely parallels that of the mouse embryo. Several elegant studies (reviewed in (Marshall and Thrasher, 2001; Peault, 1996)) have been performed to examine the origin of human HSCs. As in the mouse, prior to the onset of circulation between the embryo proper and the yolk sac, the intraembryonic splanchnopleura (from 3-4 weeks in gestation) contains cells with multilineage potential (both lymphoid and myeloid), while yolk sac contains cells with only myeloid potential (Tavian et al., 2001). Beginning precisely at 27 days of gestation (Labastie et al., 1998; Tavian et al., 1996), just prior to the establishment of liver haemopoiesis, haemopoietic cell clusters are found adhering to the ventral wall of the dorsal aorta. These cells express surface markers characteristic of haemopoietic progenitors and it is thought that these cells colonize the fetal liver and are thus, the founders of the human adult haemopoietic system (Tavian et al., 1999). Furthermore, the ventral aortic mesenchyme underlying the haemopoietic clusters resembles a haemopoietic stromal layer with a morphological cell polarity (Marshall et al., 1999). Importantly, at least part of the emerging haemopoietic cells within the human embryo appear to be derived from cells in the vascular walls (Oberlin et al., 2002). To directly analyze the haemopoietic potential of YS and AGM endothelial cells, Oberlin and colleagues purified by cell sorting, on the basis of CD31/CD34 and CD45 expression, endothelial and haemopoietic cells from these tissues. Importantly, endothelial CD31⁺/CD34⁺ CD45⁻ cells isolated from the AGM and cultured on MS-5 stromal cells give rise to haemopoietic progeny including myeloid, NK and B cells. In contrast, the sorted cells from the YS only generate myeloid and NK cells. Together, these results strongly support the existence of a hemogenic potential of the intraembryonic vascular endothelium. Similarities between haemopoietic cell emergence in the human embryo with that in the mouse embryo prompted the inventors examination of the human placenta for HSCs.

Previously, the human placenta has been examined for mesengenic cells (Wulf et al., 2004) and mesenchymal stem cells (Fukuchi et al., 2004; Romanov et al., 2003; Zhang et al., 2004) but not for haemopoietic progenitors or HSCs. Mesengenic cells were obtained from full-term placentas and propagated in vitro for three passages. These cells were exclusively of maternal origin and exhibited differentiation potential along osteogenic, chondrogenic, adipogenic and myogenic lineages and they expressed markers characteristic of mesenchymal cells: CD9, CD29 and CD73 (Wulf et al., 2004). Mesenchymal stem/progenitor cells have been isolated from placentas (Fulcuchi et al., 2004; Zhang et al., 2004) and also from the mesenchymal layer underlying the umbilical endothelium in the umbilical cord (Romanov et al., 2003). These cells displayed a fibroblastic morphology and expressed the following surface markers; CD73, CD105, CD29, CD44, HLA-ABC and CD166. They are negative for CD14, CD31, CD34, CD45 and HLA-DR. As with mesengenic cells, they differentiate to adipocytic and osteogenic lineages and additionally to the chondrogenic lineage. These cells are clearly different in both phenotype and function to HSCs and may serve as the supportive microenvironment for HSCs, providing for their growth and maintenance.

The present inventors have examined post-partum human placentas for the presence of HSCs. They have demonstrated the presence of bona fide functional HSCs in the human placenta by the SCID-NOD in vivo hematopoietic repopulation assay. This xenotransplantation assay has been used routinely as the gold-standard for identification of human HSCs in bone marrow, mobilized peripheral blood and umbilical cord cell populations.

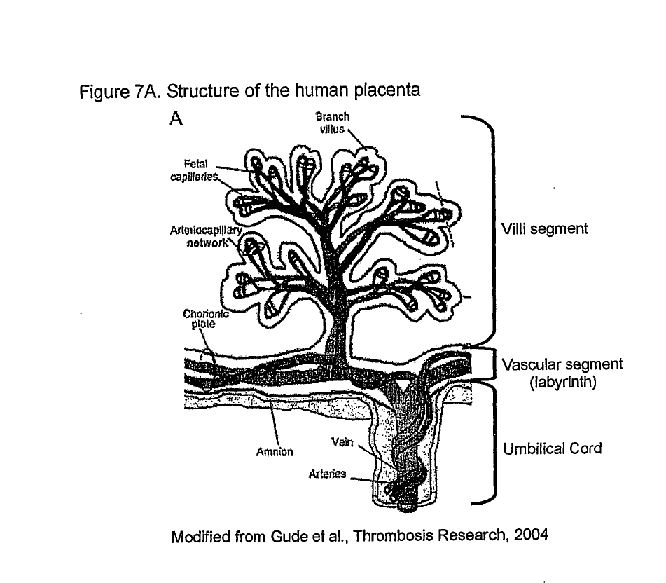

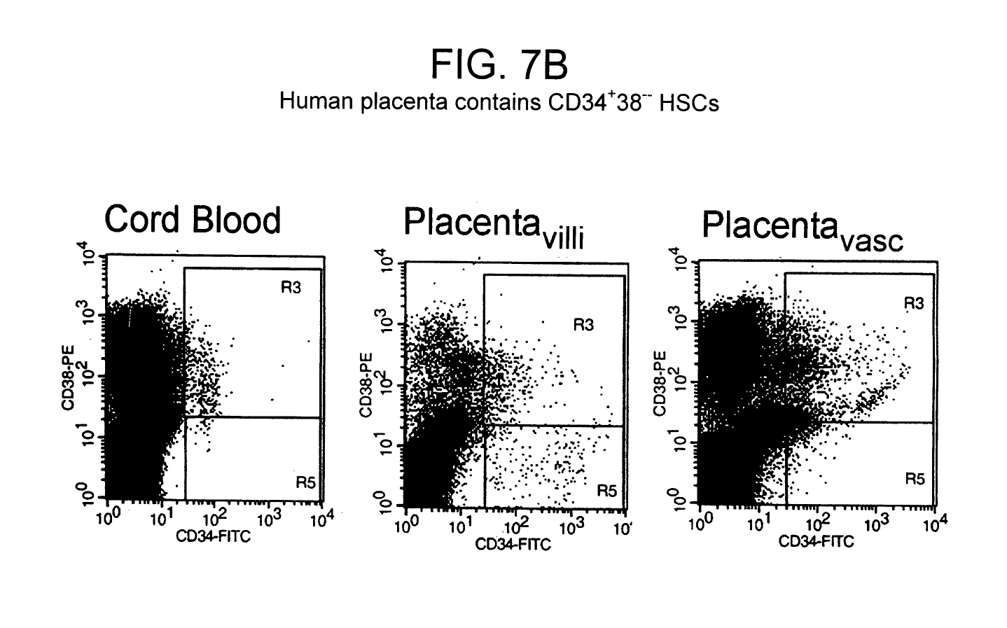

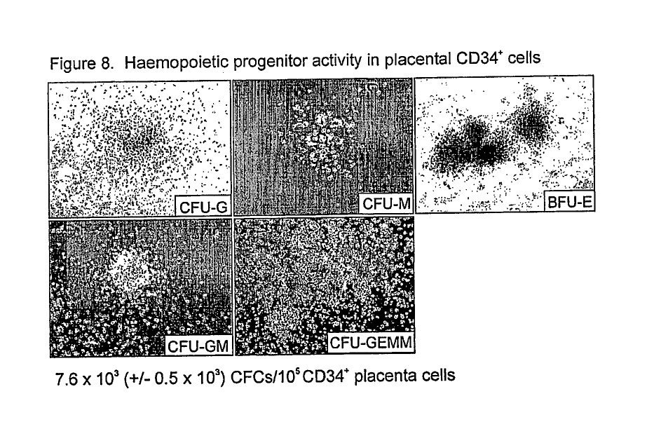

Post-partum placentas were obtained from consenting healthy individuals. During pregnancy individuals were screened to verify the absence of viral infections. Placentas were collected and processed immediately following birth. Placentas were washed extensively to remove blood remaining in the placental vessels and also to remove maternal blood contamination. Maternal tissue components were dissected away. Placental cells were dispersed by mechanical and enzymatic methods to yield a single cell suspension. In some cases, the placenta was divided into 2 segments: a segment comprised of the placental villi and a segment derived from the major placental vasculature (extensive collagenase treatment) (FIG. 7A). FACS analysis for defined HSC markers revealed high percentages of CD34⁺ and CD34⁺ 38⁻ in both placental segments (FIG. 7B). These percentages are 10 times higher than those found in umbilical cord blood, thus suggesting that the placenta contains abundant immature haemopoietic cells (progenitors and stem cells) (Table 4). CD34⁺ placental cells were sorted and tested for uni- and multi-lineage hematopoietic progenitor activity. Both the vascular and villi segments of the placenta contained CFU-G, CFU-M, BFU-E, CPU-GM and CFU-GEMM (FIG. 8) indicating that indeed the CD34⁺ fraction of placenta contains haemopoietic progenitors. All haemopoietic progenitors were found in the CD34⁺ fraction of placenta cells and are represented at a frequency of 7.6 CFU per 100 CD34⁺ placenta cells. Progenitors for both white (CFU-G, CFU-M, CFU-GM) and red (BFU-E) blood cell lineages were found, as were progenitors with potential for multiple lineages (CFU-GM and CFU-GEMM), indicating the presence of the most immature haemopoietic cells in the placenta.

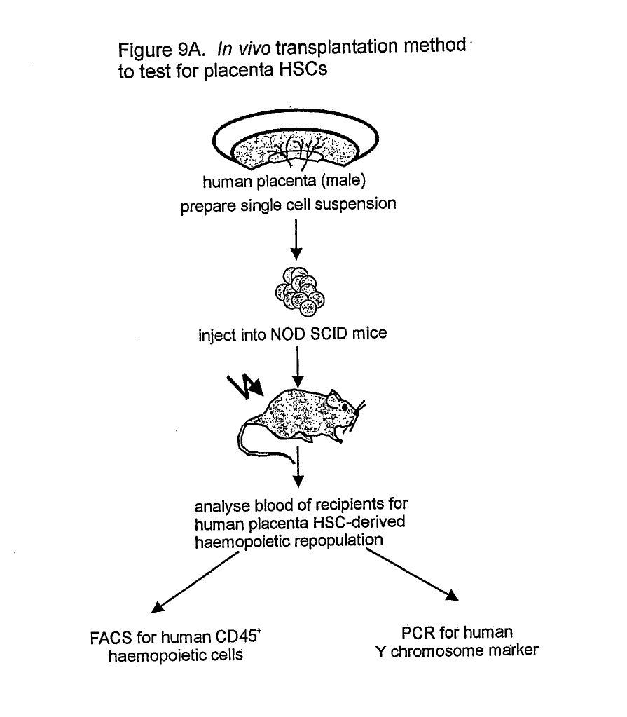

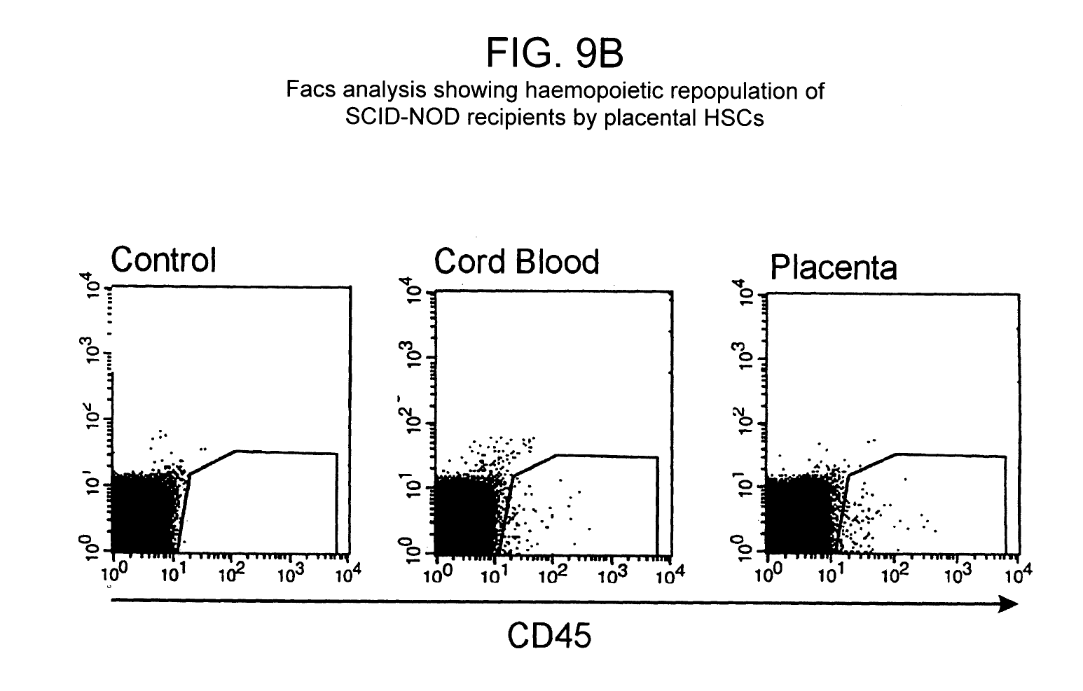

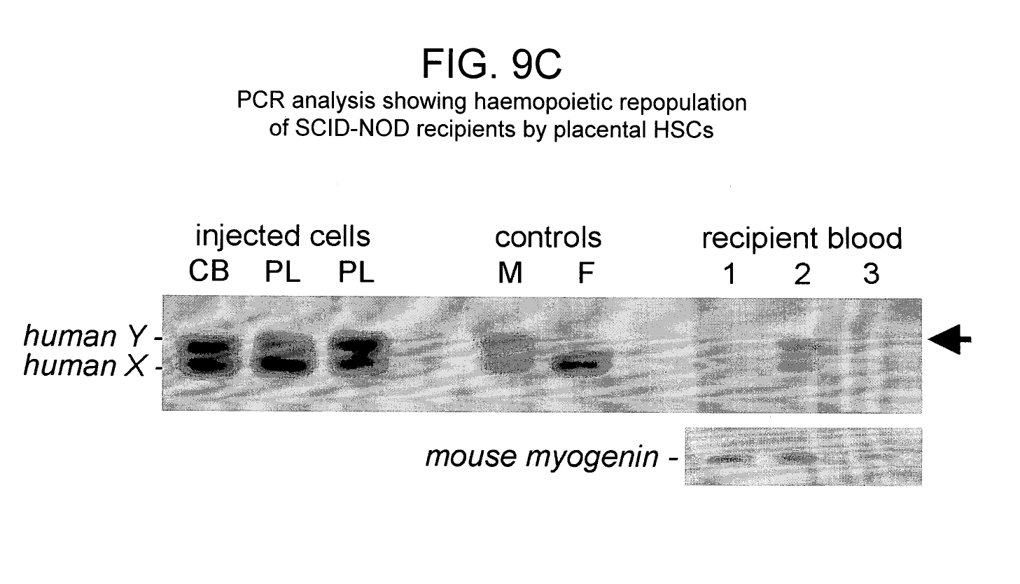

The most stringent test for human HSC function is an in vivo xenotransplatation of the putative HSC population into immunodeficient SCID-NOD mouse recipients (FIG. 9A). When placental cells were injected intravenously into sublethally irradiated SCID-NOD recipients, human CD45⁺ hematopoietic cells were observed in the peripheral blood as early as 3 weeks post-transplantation (FIG. 9B). The HSC repopulating ability of placenta was equivalent (or better) than that found in cord blood (FIG. 9B). As the placenta was male in origin, it was possible to determine whether donor male placental HSCs rather than maternal HSCs contributed to the haemopoietic repopulation. A human Y and X chromosome PCR method was used. As shown in FIG. 9C, PCR of peripheral blood DNA of female SCID-NOD mouse recipients receiving human male placental cells (lane 2) showed the presence of the human Y chromosome marker. The equimolar presence of the Y and X chromosome markers proves that the repopulating HSCs were derived human male placental cells, thus demonstrating that the placenta is a potent source of functional in vivo repopulating HSCs.

(C) Cell-Surface Markers Detected on the Surface of HSCs Isolated from Post-Partum Placenta.

The following cell surface markers have been to detect HSCs isolated from mouse and/or human placenta by the present inventors: Sca-1, CD34, CD38, CD45, c-kit³

(D) Enrichment of Haemopoietic Stem Cells Isolated from a Post-Partum Placenta.

Enrichment of HSCs isolated/extracted from post-partum placenta for therapeutic use and/or functional analysis is possible through a variety of techniques including density gradient centrifugation, flow cytometric sorting based on activation, size and/or cell cycle status and combined surface antigen expression. These methods will be familiar to those skilled in the art. However, to date no unique single phenotypic characteristic has been found to specifically isolate HSCs. Instead, sorting for several cell surface protein markers, in combination with the techniques mentioned above, is commonly used in the enrichment of HSCs.

In this context, mouse adult bone marrow HSCs are the best characterized. In addition to the absence of mature haemopoietic lineage-specific markers (Lin⁻), they express the two glycophosphatidyl inositol-linked immunoglobulin superfamily molecules, Sca-1 (Ly6A/E) and Thy-1 (at low level) (Spangrude et al., 1988) and also highly express the c-kit receptor tyrosine kinase (Ikuta and Weissman, 1992). Thus, the murine c-kit⁺Thy-1^{low}Sca-1⁺Lin⁻ bone marrow cell population clearly contains most/all HSCs. Most recently, adult bone marrow HSCs have been efficiently sorted based on their staining with rhodamine and high endoglin expression (Chen et al., 2003).

Some HSC markers are highly conserved across species barriers (such as CD34) and some vary with species (Thy-1, CD38), allele (Thy-1.1/1.2, Ly-6A/E), developmental stage and/or anatomical site of residence (Lansdorp et al., 1993; Morrison et al., 1996; Sanchez et al., 1996; Spangrude et al., 1988). Concerning spatial and temporal expression differences, adult bone marrow HSCs can be CD34⁺ or CD34⁻ (Ito et al., 2000; Ogawa, 2002; Osawa et al., 1996), while all AGM and fetal liver HSCs are CD34⁺. Thus, the molecular profile characterizing HSCs is variable, and HSCs in the embryo are somewhat phenotypically distinct from their adult counterparts due to their localization, but more likely, due to the fact that they are newly emerging cells taking on a stem cell fate.

In particular, AGM HSCs are found in close association with the major vasculature of the embryo in a unique inducing microenvironment. The developmental lineage relationship between the haemopoietic and endothelial lineage suggested by this co-localization is strengthened through molecular expression studies which reveal common expression of surface markers such as Flk1, tie-2, CD31, CD34, c-kit, AA4.1, Flt-3 ligand, VEGFR1/2, Sca-1, VCAM1 and transcriptional factors such as SCL, GATA-2, Runx1 and Lmo2 in both associated cell types in the mouse embryo (Garcia-Porrero et al., 1998; Keller et al., 1999; Nishikawa, 2001; Shivdasani et al., 1995; Zhu and Emerson, 2002). In the human embryo, CD34 and CD31 are expressed by vascular endothelial cells, haemopoietic progenitors and stem cells (Bonnet, 2002; Marshall and Thrasher, 2001). In both species CD45 is exclusively expressed by haemopoietic cells including HSCs. Thus, the differential expression of CD34 (or CD31, which is often restricted to the cells closest to the lumen) and CD45 allows the discrimination of intra-aortic haemopoietic cell clusters (CD34/31⁺CD45⁺) from adjacent endothelial cells(CD34/31⁺CD45⁻) (Garcia-Porrero et al., 1998; Jaffiedo et al., 1998; Marshall et al., 1999; Tavian et al., 1996). Human intra-aortic CD34⁺ cells in the clusters express some haemopoietic transcriptional factors (SCL, GATA-2, Runx1 and c-myb) and also many molecules involved in homing and adhesion (CD44/HCAM, WASP, CD106/V-CAM1, VE-cadherin, CD31) (Bernex et al., 1996; Garcia-Porrero et al., 1998; Labastie et al., 1998; Marshall et al., 1999). The majority of cells in the clusters are von Willebrand factor (vWf)⁻ and are BSLB4⁻ while the area underlying the haemopoietic clusters is vWf⁺. In the human fetal liver, the discrimination between endothelial and HSCs is more straightforward since CD34 expression is restricted to haemopoietic cells and CD31 to endothelial cells. It is important to remark that the expression of some endothelial markers is variable during the development and depends also on the localization and the size of vessels. For example, all endothelial cells are CD34⁺ and CD31⁺, but vWf and FGF-R expressions are restricted to the large vessels and BSLB4 to the capillaries. Several markers (CD31, vWf or lectin BSLB4) are present from the onset of blood vessel development.

The isolation of HSCs from the mouse embryo for functional studies has relied mainly on the c-kit, CD34, Sca-1 and Runx1 markers. The c-kit marker, which is commonly used to sort for adult bone marrow HSCs and haemopoietic progenitors, is expressed on 10-15% of midgestation mouse AGM cells (Sanchez et al., 1996). All midgestation AGM HSCs express c-kit at high levels as determined by in vivo repopulation with c-kit sorted cells. Since CD34 is expressed on only 25% of c-kit⁺ AGM cells, sorting based on both c-kit and CD34 expression yields further enrichment of HSCs (representing 2% of total cells). Interestingly, Mac-1, which is a mature macrophage lineage marker in the adult (and not expressed on adult bone marrow HSCs), is expressed on 50% of AGM c-kit⁺ HSCs (Sanchez et al., 1996). This expression together with the known expression of Mac-1 on all fetal liver HSCs, suggests that this marker is indicative of HSCs that migrate and colonize the fetal liver. Also, in the E11 mouse AGM, around 2% of cells are Sca-1⁺. Long-term transplantation experiments with Sca-1 sorted cells have shown that HSCs are localised in both AGM cell fractions, Sca-1⁺ and Sca-1⁻, and not only in the Sca-1⁺ fraction as expected from adult bone marrow and fetal liver sorts (de Bruijn et al., 2002). This result could be explained by a low or negative expression of Sca-1 by HSCs as they are actively being generated in the AGM region. This notion is supported by results from transgenic mice expressing the GFP reporter gene under the control of the Sca-1 (Ly-6A) gene regulatory sequences. In these mice, all adult bone marrow HSCs express the Ly-6A GFP transgene and sorting based on GFP expression (yielding 100-fold enrichment of HSCs) (Ma et al., 2002). Importantly, 2% of E11 AGM cells express GFP and, unlike the Sca-1 marker, Ly-6A GFP transgene expression marks all HSCs in the AGM region. While the Sca-1 protein on the surface of these HSCs may be limiting, the cytoplasmic fluorescence signal produced by eight copies of the Ly-6A GFP transgene allows for greater sensitivity of detection. These GFP⁺ cells are found specifically in the aortic endothelium and associated haemopoietic clusters of the AGM. Thus, the temporal and spatial expression pattern of Ly-6A GFP together with the finding that all AGM HSCs are GFP⁺ localize these first stem cells to the aortic endothelium and/or associated haemopoietic clusters in the midgestation mouse embryo. This is consistent with the expression patterns of c-kit and CD34 in the aortic endothelium and haemopoietic clusters. Furthermore, all AGM HSCs are CD31⁺, VE-cadherin⁺ and 50% are Flk-1⁺ (North et al., 2002).

Extensive sorting experiments for AGM HSCs have also been performed with embryos in which a lacZ reporter gene was recombined into the Runx1 transcription factor gene. Mice that are deficient for Runx1 have a complete block in definitive haemopoiesis, while primitive YS haemopoiesis is unaffected (Okuda et al., 1996; Wang et al., 1996). Runx1 expression in the E10.5 AGM region (as followed by the expression of a lacZ reporter in a mutant allele) is in haemopoietic cells in the lumen of the dorsal aorta, as well as in cells attached to the endothelium (North et al., 1999). Endothelial cells in the dorsal aorta are positive, the majority of which are located on the ventral side of the aorta. When sorted and transplanted into irradiated adult mouse recipients, all midgestation AGM HSCs were found to be Runx1 lacZ⁺ (North et al., 2002). Hence, the HSCs can be efficiently isolated based on the expression of several cell surface and molecular markers in limited number of cells localized to the endothelium and haemopoietic clusters of the embryo.

(E) Enrichment of Haemopoietic Stem Cells Isolated from a Post-Partum Placenta.

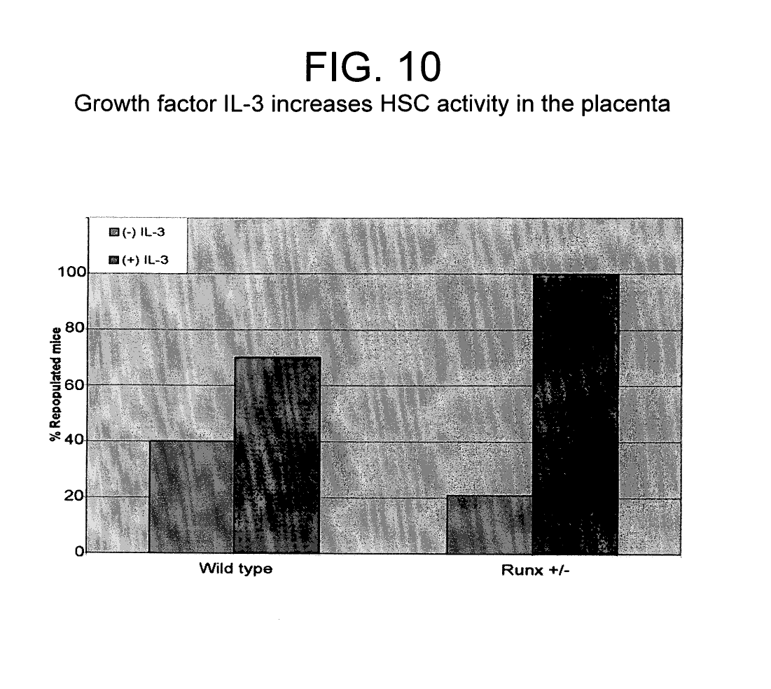

Several culture systems offer the opportunity of growth and expansion of HSCs. Explant cultures of whole AGM tissue increase HSC activity by 16-fold within 3 days (Medvinsky et al., 1996). Explant cultures of placenta may be used to expand placenta HSCs. Alternatively, stromal cells isolated from the AGM and/or placenta may be used to expand placental HSCs. The addition of cocktails of haemopoietic growth factors are also useful in such expansion cultures and may include, IL-3 (Table 3), EL-6, Tpo, OSM, SCF, GM-CSF, MIP1γ, Wnt, BMP, NGFβ (FIG. 6). The inventors have shown that placental HSCs can be increased by ex vivo culture in hematopoietic growth factor IL-3. Mouse placental tissues were cultured as explants for 3 days in the absence or presence of L-3. Cells from these ex vivo treated placentas were injected into irradiated adult recipient mice to test for HSC repopulation. Donor cell repopulation was compared. As shown in FIG. 10, ex vivo IL-3 treated placentas repopulated more recipients. Indeed, culture in IL-3 increased HSC activity 1.8- to 5-fold. Hence, the ex vivo treatment of placental tissue offer great opportunity for the growth and expansion of potent HSCs.

### (F) Post Partum Human Placenta Haemopoietic Stem Cell Storage

Cord blood banks have been established in which human cord blood haemopoietic cells are stored in defined freezing medium by well established protocols under GMP conditions. Cells are stored indefinitely in liquid nitrogen and have been shown to retain HSC activity. The inventors have tested for the maintenance of human CD34⁺ placental cells and have found that such cells remain viable, are functional and are even enriched after storage (Table 5). Thus, placental HSCs are excellent candidate stem cells for banking and future use in clinical therapies.

### (G) Significance of the Present Invention.

The present inventors have shown here that the placenta, a highly vascularized tissue necessary for the exchange of oxygen and nutrients between the embryo and the mother, contains potent adult repopulating HSCs. These are not maternally-derived HSCs, but are derived from the conceptus, as evidenced by the presence of the human β-globin and Ly-6A GFP transgenic markers in the mouse (Table 1) and human male Y chromosome marker positive cells in SCID-NOD transplanted mice (FIG. 9C). Moreover, the percentages of HSCs (as defined by CD34⁺38⁻phenotype) are higher in the human placenta than in cord blood (Table 4). They are as potent as HSCs derived from the adult bone marrow in that they give rise to long-term, high level multilineage haemopoietic engraftment of irradiated adult recipients. Placental HSCs are also self-renewing, since they can repopulate secondary recipients. These novel data in the mouse and human species now provide evidence that along with the AGM and YS, the placenta is an additional generating source of HSCs that sequentially migrate and colonize the fetal liver and bone marrow (FIG. 5). Moreover, placental HSCs can be increased by ex vivo treatment with hematopoietic growth factors. Placental HSCs can be stored and maintained in their viability and function for later use in transplantations. Hence, placenta HSCs offer great benefit for banking and use in clinical transplantations and cell replacement therapies.

### The Placenta as a Reservoir for Expanding HSCs or for Their Generation?

The data provided by the inventors shows that prior to mouse E11, placental HSC activity appears to be limiting. No HSCs were found in the mouse placenta at E9 or E10 (Table 1). At E11, only one out of five transplanted recipients receiving two tissue equivalents of placental cells is HSC engrafted. At E12, 12 HSCs per placenta were found and these numbers increase thereafter. Thus, the numbers of HSCs in the mouse placenta increase rapidly with developmental time. Indeed, large numbers of immature progenitors have been found previously in the mouse placenta and greatly exceed the numbers found in the fetal liver (Alvarez-Silva et al., 2003). Here, the inventors have shown that the post-partum human placenta contains potent HSCs that can functionally repopulate irradiated recipients. Others have shown that the human placenta secretes haemopoietic growth factors that stimulate haemopoietic colony formation (Burgess et al., 1977). More recent work demonstrates that mesenchymal progenitor cells isolated from the human placenta can expand long-term culture-initiating cells from cord blood (Zhang et al., 2004). Hence, the haemopoietic growth capacity of the placenta is enormous demonstrating that it is a potent haemopoietic microenvironment for HSCs.

Recently, quantitative data on the numbers of HSCs present in the various haemopoietic tissues and circulation in the midgestation mouse has been presented. Kumaravelu and colleagues found that at E12 there are 3.2 HSCs in circulation (Kumaravelu et al., 2002). Correcting for numbers of HSCs in tissues due to circulation, the mouse AGM was found to contain 2.7 HSCs, the YS 1.8, the liver 53 and the umbilical cord 0.8. Given that the E12 mouse placenta contains 12 HSCs (four times more HSCs than in the whole of the embryonic blood) and considering that the fetal liver contains 53 HSCs at E12, it is highly likely that the placenta is a potent HSC contributor to the colonization of the fetal liver, along with the AGM and YS.

It is as yet uncertain whether the HSCs in the placenta are intrinsically or extrinsically generated. If the placenta was found to contain HSCs at E10 or earlier, it would implicate the placenta as the first site of emergence of HSCs in the mouse embryo. Previously, to detect the onset of HSC activity in the E10 AGM region, it was necessary to transplant 96 adult recipients with a total of 112 AGM tissue equivalents of cells to observe the long-term, high level, multilineage repopulation of 3 recipients (Muller et al., 1994). Given the limited number of mice transplanted with E10 placental cells in this study, it remains a possibility that the placenta contains HSCs at this earlier time point. Thus, the origins of the HSCs in the placenta are unclear and await the results of lineage marking approaches.

### Association of Placental HSCs and the Vasculature

The strong association of haemopoietic and endothelial markers on the earliest haemopoietic cells in the mouse embryo, including HSCs, prompted the present inventors' studies on the placenta. The Ly-6A GFP transgenic marker was found to be expressed in the endothelial cells lining the major vessels of the placenta. Moreover, all HSC activity was attributed to the GFP⁺ fraction of the placenta. Thus, consistent with previous data, the Ly-6A GFP expression again marks all HSCs. While the number of GFP⁺ cells in the placenta far exceeds the number of HSCs, other markers (such as CD34, CD31, c-kit, etc.) were used to further localize placental HSCs. Previous studies on the AGM have shown that all AGM HSCs are c-kit⁺CD34⁺ (Sanchez 1996) and CD31⁺ (North et al., 2002). The inventors' multicolor flow cytometric analysis strongly suggests that placental HSCs are GFP⁺c-kit⁺CD34⁺ (Table 2).

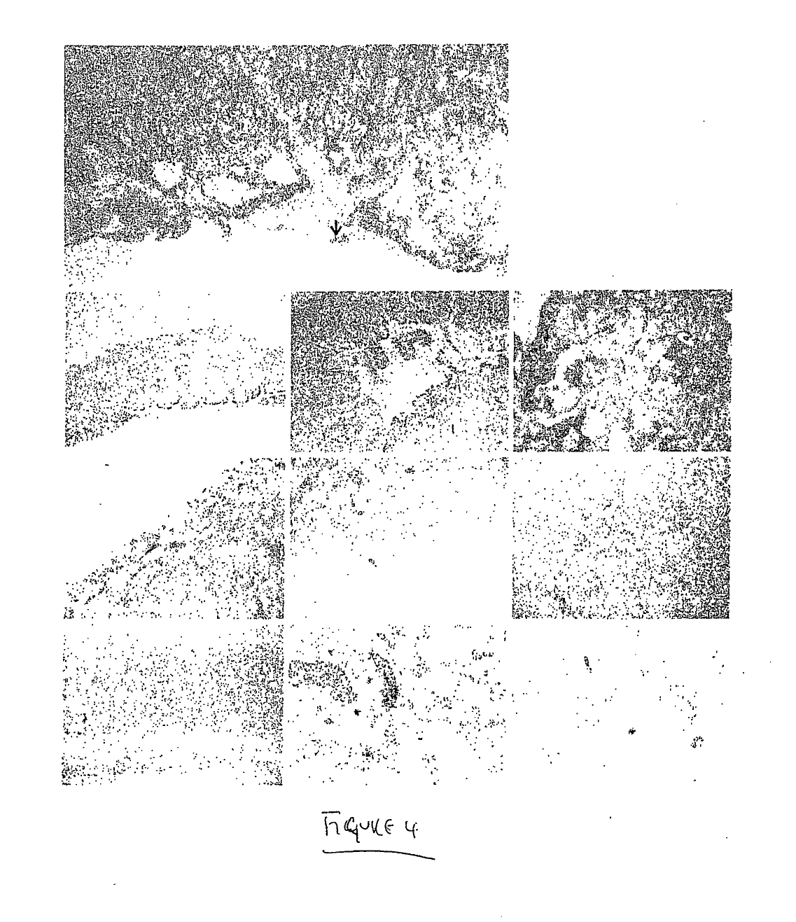

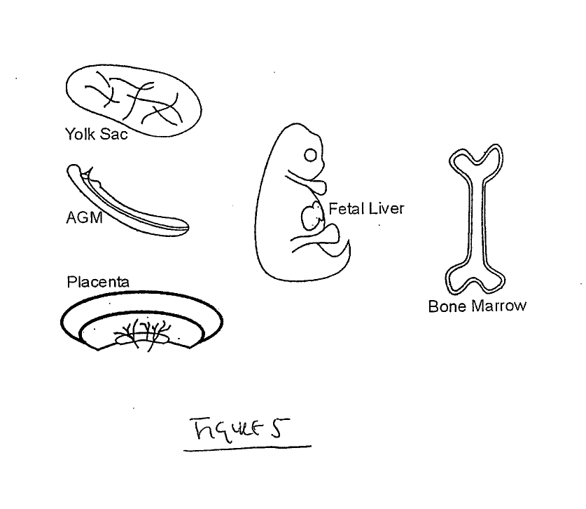

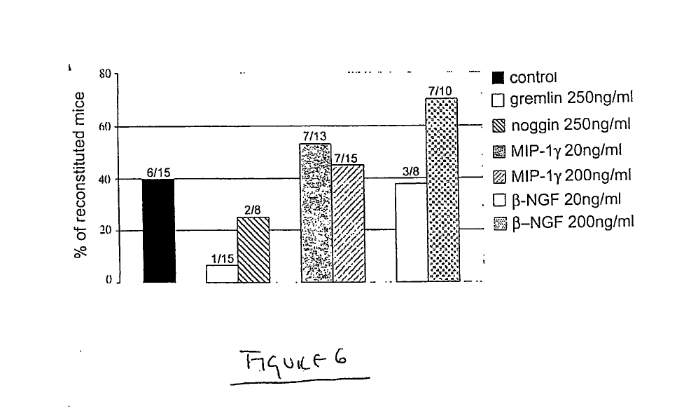

Immunostaining of Ly-6A GFP transgenic placentas has further supported the notion that these HSCs are CD34⁺. CD34 and GFP co-expressing cells were found in the labyrinth region (FIG. 3). This highly vascularized region showed co-expressing cells lining the embryonic vessels. Similarly, although at a lower frequency, CD31 and GFP co-expressing cells were also found in endothelial cells lining the vessels of the labyrinth. Moreover, c-kit expression was found in a few cells in this region by in situ transcription analysis (FIG. 4A). In no sections did the inventors find prominent haemopoietic clusters with these phenotypic characteristics. However, they did observe rare single GFP⁺ cells adhering closely to the lumenal surface of the larger vessels. Thus, in analogy to the AGM region in which GFP and CD34/CD31 are co-expressed in the ventral aortic endothelial cells and some cells of the associated haemopoietic clusters, these markers may also indicate associations of these two lineages in putative hemogenic endothelium of the placenta. Similarly, the human CD34 marker (FIG. 7B) suggests associations between hemogenic endothelium and HSCs in the human placenta.

A role for Transcription Factors GATA2 and Runx1 in Placental Haemopoiesis?

Previously, it was reported that GATA2 and GATA3 are highly expressed in the placenta in the trophoblast giant cells positioned at the embryonic-maternal interface during midgestation (Ng et al., 1994). The GATA transcription factors regulate the expression of a number of trophoblast-specific genes, such as the prolactin hormone placenta lactogen I and the angiogenic factor proliferin (Ma et al., 1997; Ng et al., 1994). These molecules appear to play an important role in the neovascularization of the placenta in the interface region (Ma et al., 1997). The present inventors also found expression of these transcription factors in trophoblast giant cells. The lower frequency of GATA3 expressing trophoblast cells (FIG. 4E-F) that they found most likely was a reflection of a haploinsufficiency of GATA3 due to a defective targeted allele. Nonetheless, GATA3 was restricted in its expression to these cells and is known to rapidly decline in its expression after E10 (Ng et al., 1994).

GATA2 had a much more widespread expression pattern than previously described (FIG. 4B-D). GATA2 was expressed in some endothelial cells lining the vessels of the labyrinth, as well as in many cells surrounding the vessels. The intensity of expression increased with proximity to the chorionic plate of the placenta. At the interface of the labyrinth region with the chorionic plate, GATA2 expression was the highest. Thus, GATA2 may be required for the neovascularization of the labyrinth by regulating the expression of angiogenic factors. Additionally, it may also be involved in the generation and/or proliferation of HSCs from hemogenic endothelial cells in the labyrinth.

Previously, the expression of Runx1 in the placenta was found by RT-PCR (Alvarez-Silva et al., 2003), but its expression pattern within the placenta was not explored. As one of the most important HSC transcription factors, Runx1 is required for the emergence of HSCs in the midgestation embryo and is expressed in aortic haemopoietic clusters, endothelial cells and underlying mesenchymal cells (North et al., 1999). The expression pattern of Runx1 lacZ in the placenta is reminiscent of this pattern (FIG. 4G-J). The present inventors found high-level Runx1 expressing cells in the lumen of the labyrinth vasculature as well as in some of the endothelial cells lining the vessels. Lower level expressing cells were found underlying some of the endothelium. Thus, as in the AGM region, Runx1 is most likely playing a role in the emergence of HSCs.

Finally, we have also investigated the possible role of the oncogene c-fos in placental HSC generation. High levels of c-fos have previously been observed in the placenta (Muller et al., 1983) and a placental defect in c-fos^{-/-} embryos been suggested on the basis of lower placenta weight, although no conclusive evidence had been provided (Johnson et al., 1992). The inventors transplanted E12 placentas from c-fos^{-/-} embryos and found some low level HSC activity still present (data not shown).

In summary, the present inventors have found that mouse and human placentae are highly haemopoietic tissues, supporting the growth and/or emergence of potent in vivo repopulating HSCs. As found by flow cytometric and histologic analysis, the placenta appears to possess hemogenic endothelium. Notwithstanding, this may be taken as another example of the recurring theme of HSC development within the major vasculature of the embryo and indicates that the Ly-6A GFP transgene serves as a useful marker for hemogenic endothelium in the embryo. Thus, the results now add the placenta to the list of embryonic tissues that contain HSCs (FIG. 5) and it is proposed that it plays an important role in the long-term development of the adult haemopoietic system and the colonization of the bone marrow with HSCs.

The invention will now be described by way of the following examples which in no way be considered limiting of the invention.

### EXAMPLES

### Example 1

### Materials and Methods

Human placentas are collected from normal full-term pregnancies (consenting donors) according to regulations of Erasmus MC and the Medical Ethical Board. Extra tissues such as amniotic sac and decidua and other maternal components are removed from the placenta. The whole placenta as well as the vascular labyrinth is extensively washed in phosphate buffered saline (PBS) containing EDTA. Medium containing 0.125% type I collagenase (or other enzymes such as dispase or trypsin) is injected inside the vascular labyrinth to detach closely adherent and emerging haemopoietic cells and hemogenic endothelium. The injected placenta is incubated for 1 hour at 37° C. After incubation, the solution is collected and the labyrinth is washed extensively. The wash is pooled and the cells are centrifuged at 1000 rpm for 5 minutes. Mononuclear cells are collected after density gradient centrifugation in Ficoll or Percoll.

Villi segments from freshly delivered human term placentas are dissected in cold PBS, minced, passed through a stainless steel large mesh grid. Cells are washed thoroughly with cold Hank's balanced salt solution (HBSS) or phosphate buffered saline (PBS) containing EDTA. The residual tissue fragments are then treated with 0.125% type I collagenase (Sigma-Aldrich Chemie, Gmbh, Germany) in PBS (or medium 199) supplemented with 10% fetal calf serum (or other serum-free supplements) and penicillin/streptomycin (or other suitable antibiotics) for 1.5-2 hours. The tissue is then dissociated by repeated pipetting and transferred to centrifuge tubes. After sedimentation, the suspended cells are transferred to a fresh tubes and the remaining pellet of large fragments washed several times with cold PBS (or HBSS) supplemented with 10% fetal calf serum (or other serum-free supplements) and penicillin/streptomycin (or other suitable antibiotics). The suspended cells from each wash are pooled. Smaller cell clumps are further treated with enzymatic digestion (for example 0.2% trypsin/0.1% Dnase type IV) for maximum yield of suspended cells. The pooled cells are then filtered through a course sterile cotton mesh and/or a 56-100 μm nylon mesh. The large number of erythroid cells are removed by subjecting the cell suspension to a density gradient centrifugation. These steps already yield a high level of enrichment.

Viable cell counts are determined by trypan blue exclusion prior to further enrichment procedures, assay or storage.

### Exclusion of Other Cell Placental Cell Types.

Various populations of cells in the placenta may be negatively selected. These include mesenchymal stem cells (MSC) and cytotrophoblast cells. Cytotrophoblast cells may be eliminated by the method of Kliman et al (Kliman et al., 1986). Human placental mesenchymal stem/progenitors cells may be eliminated through antibody mediated selection based on cell surface markers expressed on these cells. Maternal cells may also be excluded or fetal cells enriched by this method. These may include antibodies directed against antigens such as CD73, CD29, CD44, HLA-ABC. One or more of these markers can be used in a negative selection procedure that eliminates these cells from the placental cell pool. These antibodies may be used for negative selection by magnetic-bead, panning or flow cytometric methodologies. An additional, pre-enrichment step may include adherence to plastic or to reagent coated plates. For example, this more simple approach eliminates fibroblastic type cells.

### Positive Selection of Cells Including Placental Haemopoietic Stem Cells.

Because of the shared phenotype of endothelial and haemopoietic stem cells and for optimal isolation of the clinically relevant repopulating stem cells within both populations, enrichment processes aim at the isolation of both of these cell lineages. As endothelial cells require further treatments in order to disrupt their strong association within the endothelium and to obtain single cell suspensions, tissue preparations may be further subjected to enzymatic perfusion in order to release endothelial cells from small placental vessels. Further purification steps then include centrifugation in density gradients to isolate mononuclear cells. The final step in the enrichment for repopulating HSCs may be specific antibody-mediated cell sorting, panning or plate adherence procedures. Initially, HSCs will be separated from mature haemopoietic cells by the negative selection of lineage marker (mature haemopoietic cell markers such as CD19, CD8, CD4, CD15, CD11b, CD56)-expressing cells, as these markers are not found on HSCs. Cocktails of antibodies against lineage markers may be used to remove mature haemopoietic cells in a single step. As a positive enrichment step HSC activity may be obtained in the CD34⁺CD38⁻ and CD34⁺CD38⁻ population. Alternatively, the side population and more specifically the tip of the side population of Hoescht 33342 stained cells may yield a highly enriched HSC population.

### Enhancement of Placental HSC Growth and Expansion.

Several culture systems offer the opportunity of growth and expansion of HSCs. Explant cultures of whole AGM tissue increase HSC activity by 16-fold within 3 days (Medvinsky et al., 1996). Similar cultures of placenta may be used to expand placenta HSCs. Alternatively, stromal cells isolated from the AGM and/or placenta may be used to expand placental HSCs (FIG. 6). The addition of cocktails of haemopoietic growth factors are also useful in such expansion cultures and include, IL-3 (Table 3), IL-6, Tpo, OSM, SCF, GM-CSF, MIP1γ, Wnt, BMP, NGFβ (FIG. 6).

### Example 2

### Results

### Expression of Ly-6A GFP in the Highly Vascularized Tissues of the Embryo

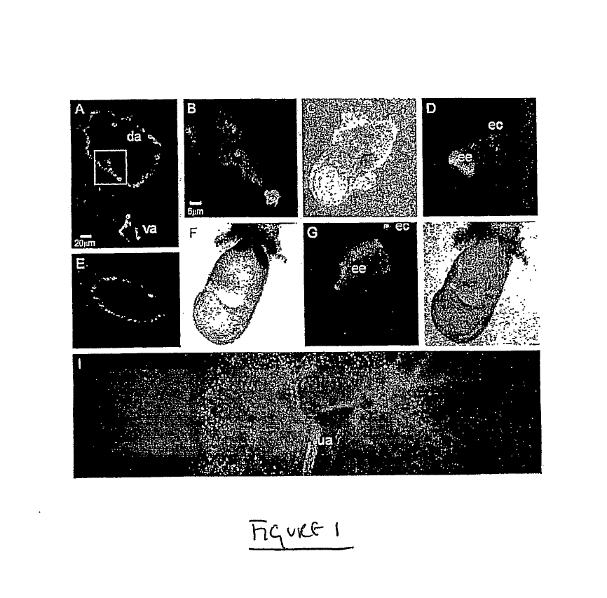

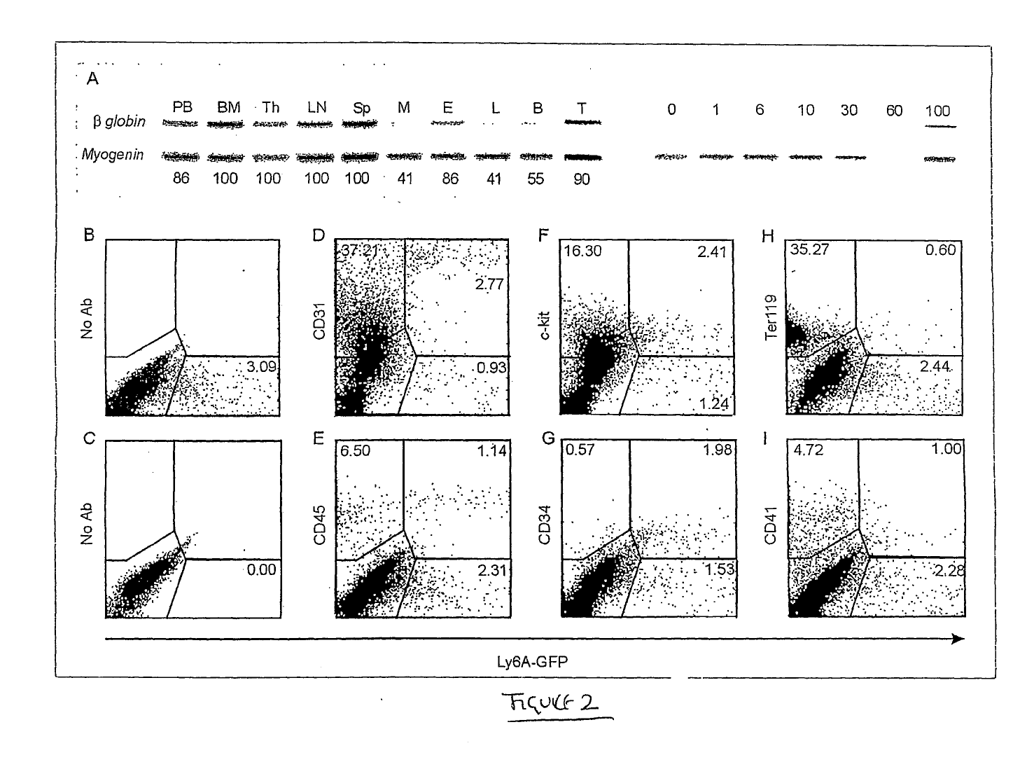

Previously the inventors have shown that all HSCs in the AGM region of the midgestation mouse embryo are localized to the Ly-6A GFP⁺ aortic fraction of cells (de Bruijn et al., 2002). GFP expression is found in some endothelial cells of the dorsal aorta and an even stronger expression is found in the endothelial cells lining the vitelline artery (FIG. 1A). Haemopoietic clusters along the lumen of the aorta and vitelline arteries also contain GFP⁺ cells (FIG. 1B). Moreover, strong GFP expression is found in the umbilical artery (FIG. 1E) and the major vessels of the YS (de Bruijn et al., 2002). All these sites contain potent HSC activity as determined by in vivo transplantation into adult irradiated recipients (de Bruijn et al., 2000; Medvinsky et al., 1996).

In addition to these known haemopoietic tissues, the placenta is also highly vascularized and thus, the inventors examined this tissue for expression of the Ly-6A GFP marker. As the placenta is formed from both maternally and embryonic-derived cells, the Ly-6A GFP transgene was transmitted only through the male germline. Already in the pre-streak stage embryo, high GFP expression is found in the extraembryonic ectoderm and ectoplacental cone (FIGS. 1C, D). The highest intensity of GFP expression is found where the extraembryonic ectoderm borders the primitive ectoderm. At E7.25, the extraembryonic ectoderm remains GFP⁺ (FIG. 1F-1H). At this stage the exocoelom is forming and the extraembryonic ectoderm appears to be advancing towards the GFP⁺ ectoplacental cone. Most strikingly, at E12 high levels of GFP expression are found in the embryonic vessels of the placenta (FIG. 1I). Thus, similar to the other major haemopoietic tissues in the embryo, the placental vasculature expresses Ly-6A GFP suggesting a potential for haemopoietic activity.

### Placenta Contains Potent HSCs Beginning at Midgestation

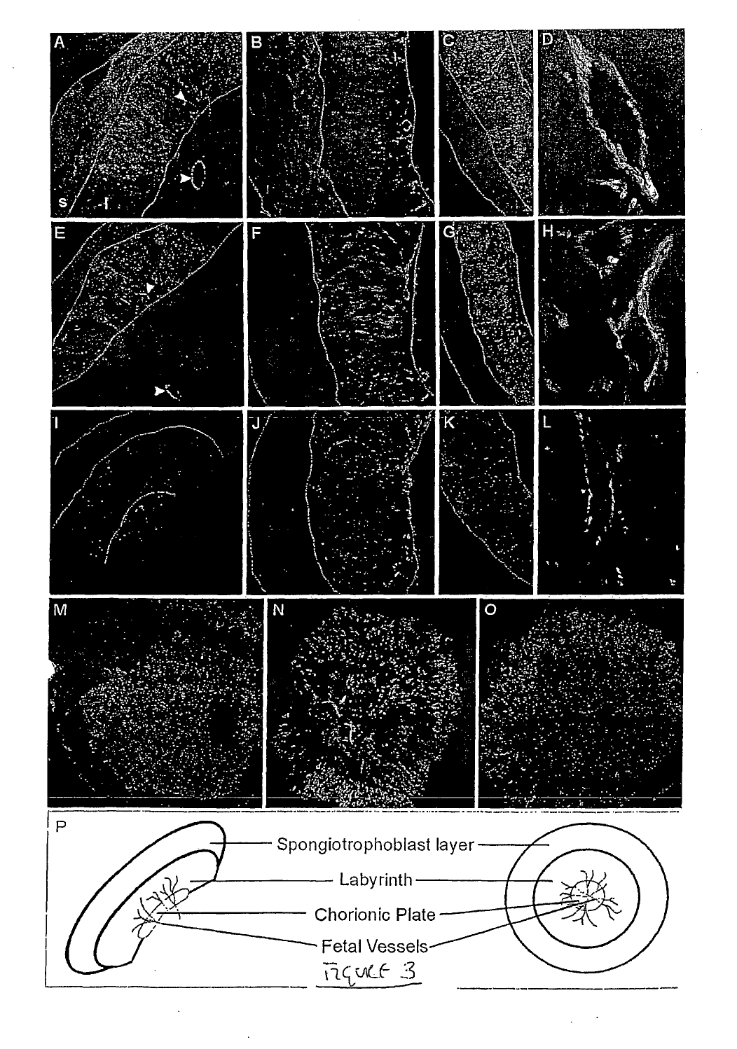

Previously, Alvarez-Silva and colleagues have found haemopoietic progenitor activity in the placenta (Alvarez-Silva et al., 2003). However, the presence of HSC activity was not tested. To examine the placenta for potent adult engrafting HSCs, the inventors obtained E9 though E12 embryos (human β-globin transgenic), dissected the placenta (without the decidua or umbilical vessels), made a single cell suspension and injected various doses of cells (placental tissue equivalents) into irradiated adult recipients. Engraftment was tested by peripheral blood DNA PCR for the presence of the donor (human β-globin) marker at 1 and greater than 4 months post-transplantation. As shown in Table 1, no engrafted mice were found after transplantation of E9 or E10 placenta cells. However, potent engraftment (greater than 10% donor cell contribution) was found beginning at E11 and was present at high levels at E12. The inventors tested these recipients for multilineage engraftment. High-level engraftment was observed in all haemopoietic tissues and in sorted myeloid, T lymphoid and B lymphoid cells in recipients of E11 (not shown) and E12 placental cells (FIG. 2A). Moreover, the transplantation of the bone marrow from these primary recipients into secondary adult irradiated recipients resulted in similar high-level repopulation at greater than 4 months post-transplantation (6 positive/6 injected, range of repopulation 75-98%). Frequency analysis of HSCs within the E12 placenta showed 1 HSC per 49,713 cells with approximately 12 HSCs per placenta (as determined by Poisson statistics). Thus, the placenta contains potent repopulating cells that fulfill all the established functional criteria of HSCs.

### All Placental HSCs are Contained Within the GFP⁺ Fraction

The inventors next performed flow cytometric analysis to determine the number and phenotypic characteristics of GFP⁺ cells in the midgestation placenta. E12 placenta cells were stained with antibodies specific for CD31 (endothelial, macrophage and AGM HSC marker (North et al., 2002)), CD34 (endothelial and AGM HSC marker (Sanchez et al., 1996)), c-kit (HSC and immature haemopoietic progenitor marker (Sanchez et al., 1996)), CD45 (pan-haemopietic marker (Morrison et al., 1997)), Ter119 (erythroid progenitor marker (Kina et al., 2000)) and CD41 (haemopoietic progenitor and megakaryocyte marker (Mikkola et al., 2003)). GFP⁺ cells (FIG. 2B) were found to represent 3.09% of the total viable, nucleated cell population (absolute number per placenta=1.8×10⁴ GFP⁺ cells). No GFP⁺ cells were detected in the maternally-derived decidua (FIG. 2C). This was due to the paternal transmission of the transgene and confirms the embryonic origins of GFP⁺ cells. In addition, when E12 decidua cells were transplanted, no HSC activity was detected (1 month post-translation). Not surprisingly, the placenta is 36% erythroid as determined by Ter119 staining (FIG. 2H) and appears to have a high endothelial cell content as judged by the fact that 40% of placental cells are CD31⁺ (FIG. 2D). However, CD31 also marks cells of the trophoblast lineage (Cross et al., 2003a). Non-erythroid haemopoietic cells make up 5.7-7.6% of the placenta (CD41 and CD45 positive cells; FIGS. 2I and E, respectively). There is almost no overlap in Ter119 and Ly-6A GFP expression. However, 7%, 13%, 15% and 78% of the respective CD31⁺, c-kit⁺, CD45⁺ and CD34⁺ populations are Ly-6A GFP⁺. Most interestingly, 75%, 66%, and 56% of GFP⁺ cells express CD31, c-kit (FIG. 2F) and CD34 (FIG. 2G), respectively.

Since AGM HSCs coexpress c-kit and CD34, the inventors tested the overlap in expression of these markers with GFP in placental cells. As shown in Table 2, over 50% of GFP⁺ cells are c-kit⁺CD34⁺. Similarly, over 50% of GFP⁺ cells are c-kit⁺CD31⁺, while lower percentages of GFP⁺ cells are c-kit⁺CD41⁺ and c-kit⁺CD45⁺. This marker distribution within the GFP⁺ placental cell population is reminiscent of that observed for AGM HSCs (de Bruijn et al., 2002) and thus, suggests that at least some of the placental GFP⁺ cells are HSCs.

To determine whether the placental HSC activity lies within the GFP⁺ fraction of cells, the inventors sorted GFP⁺ and GFP⁻ cells from E12 transgenic placentas. One and 0.25 placenta equivalents of cells were injected into irradiated adult recipients and were analyzed I and/or 4 months later for donor cell engraftment. As shown in Table 1, all HSC activity was found in the GFP⁺ fraction. These cells yielded high level, long-term repopulation and contributed to all haemopoietic lineages (not shown). In addition, secondary transplantations of bone marrow showed that GFP⁺ placental HSCs are self-renewing (4 positive/6 injected, 1 month post-transplantation). Thus, all placental HSCs are Ly-6A GFP expressing. However, since frequency analysis demonstrates that there are only approximately 12 HSCs per E12 placenta, not all GFP⁺ cells are HSCs. These results are consistent with previous findings in the AGM region for GFP⁺ cells.

### GFP⁺ Cells Localize to Vascular Labyrinth

Since flow cytometric analysis demonstrated that GFP⁺ cells were distributed between the cell fractions characterized by both endothelial and haemopoietic stem/progenitor markers, the inventors took a temporal and spatial immunostaining approach to examine the specific localization of these cells. The inventors stained Lv-6A GFP transgenic placentas from E9 (not shown) and E10 to E12 embryos (FIG. 3) with antibodies specific for CD31, CD34 and CD41. For orientation purposes they show a schematic diagram of a transverse and coronal section of the placenta (FIG. 3P). Distinct differences in the expression patterns of the three surface markers were clearly observed in both coronal sections (FIGS. 3M-O) and transverse sections (FIGS. 3A-C, E-G and I-K). CD31 is expressed in many cells of the outer placenta spongiotrophoblast layer (FIGS. 3A-C and M). It is highly expressed on the cells of the dilated maternal blood vessels and at a lower level on endothelial cells in the labyrinth. It is also expressed by a few endothelial cells lining the vessels in the chorionic plate (FIG. 3D). Expression begins in the outer layer at E9 and by E12 is found also in the inner placenta, although at lower levels. CD34 is expressed exclusively in the cells of the inner placenta (FIGS. 3E-G, N) where it seems to outline the embryonic vessels (FIG. 3I). Expression is low at E9 and E10 and increases thereafter. In complete contrast to CD31 and CD34, CD41 shows a punctate expression pattern mainly in the inner placenta (FIGS. 31-K, O) where it predominantly marks cells within the blood vessels (FIG. 3L).

In situ hybridization was performed to localize c-kit expressing cells in the placenta, since high background staining was observed with c-kit specific antibodies. As shown in FIG. 4A, c-kit expression is found in mesenchymal cells of the chorionic plate (black arrow) and in islands of mesenchymal cells in the labyrinth (white arrows). Many of the vessels also contained c-kit-expressing endothelial cells (arrowheads). The inventors also detected expression in some trophoblast giant cells (not shown). The expression of c-kit was highest on the embryonic face of the placenta.

The general expression pattern of Ly-6A GFP is very similar to that of CD34 as expected from the results of the flow cytometric analysis (78% of CD34⁺ cells are GFP⁺). Expression begins at E9 in some of the cells of the labyrinth and increases thereafter. GFP⁺ cells are also found lining the fetal blood vessels in the chorionic plate and in the umbilical vessel (arrowheads in FIGS. 3A and E). Most overlap in the expression of GFP with CD34 is in the labyrinth region (FIG. 3F) in the endothelial cells lining the fetal vessels that form a network through this region. There appears to be only a small amount of overlap of CD31 with GFP expressing cells confirming flow cytometric data (only 7% of CD31⁺ cells are GFP⁺). These cells are found in the large embryonic vessels within the chorionic plate and also in the umbilical artery (FIGS. 3A and D and FIG. 1E). They observed no overlap in the expression of CD41 and GFP (FIGS. 3J and L). Thus, these immunostaining and in situ transcription results taken together with the multicolor flow cytometric analysis and the finding that all HSCs are GFP⁺, strongly suggest that placental HSCs are localized within the endothelium of the embryonic vessels in the chorionic and labyrinth regions.

### Placental Embryonic Vessel Endothelium Express Haemopoietic Transcription Factors

To further explore whether HSCs in the placenta may be generated in situ, the inventors examined the expression pattern of three haemopoietic transcription factors; GATA2, GATA3 and Runx1 known to be important for HSC/progenitor development. Briefly, embryos deficient for GATTA2 die at E10.5, display severe anemia and lack HSCs (Tsai et al., 1994). A haploid dose of GATA2 results in defective HSC expansion in the AGM (Ling et al., 2004). The disruption of the related transcription factor GATA3, leaves YS erythropoiesis largely unaffected, but results in defective fetal liver haemopoiesis, as demonstrated by a reduction in haemopoietic colony formation (Pandolfi et al., 1995). Runx1 deficiency results in E12.5 lethality, fetal liver anemia and an absence of HSCs (Ouda et al., 1996; Wang et al., 1996). A haploid dose of Runx1 disrupts the normal pattern of HSC emergence in the embryo (Cai et al., 2000).

The inventors obtained E11 GATA2 lacZ, GATA3 lacZ and Runx1 lacZ embryos and stained and sectioned the placentas. GATA2 lacZ embryos carry a lacZ reporter transgene that recapitulates the endogenous GATA2 gene expression pattern (Zhou et al., 1998). GATA3 lacZ and Runx1 lacZ embryos contain an endogenous gene targeted lacZ reporter (North et al., 1999; van Doorninck et al., 1999).

The expression pattern of all three transcription factors differed. In GATA2 lacZ transgenic placental sections, they found some expression in trophoblast giant cells, as reported previously (arrowhead in FIG. 4B). However, higher levels of β-galactosidase staining were found in the labyrinth (FIGS. 4B and C). An increasing intensity of staining was observed towards the fetal side of the placenta, especially on the borders of the labyrinth region with the chorionic plate. GATA2 is expressed in some endothelial cells and in the underlying cells that surround the fetal blood vessels (FIG. 4D). Like GATA2, GATA3 is expressed in the trophoblast giant cells, although to higher levels (FIGS. 4E and F, arrowheads). In contrast to GATA2, GATA3 expression is restricted to only these few cells at the fetal-maternal interface. This pattern of expression confirms the previous pattern seen by others using in situ transcription analysis (Ng et al., 1994). The endothelial expression of GATA2 (but not GATA3) is reminiscent of the expression of GATA2 at the onset of HSC emergence in the midgestation aorta (Minegishi et al., 1999; Zhou et al., 1998).

Runx1 expression appears to localize to cells within the blood vessels of the labyrinth (in the circulation, as well as cells attached to the lumenal side of the endothelium), to endothelial cells (FIGS. 4H-J) and to cells located just underneath the endothelium (arrows in FIG. 4I). Occasionally, the inventors see clusters of β-galactosidase positive cells within the circulation or attached to the endothelium. There also seems to be an accumulation of positive cells in the chorionic plate (FIG. 4G). These Runx1 expressing cells are located both within the walls of the vessels and surrounding the major blood vessels at their junction with the umbilical vessels (FIG. 4G). Thus, the distribution of Runx1 expressing cells amongst haemopoietic, endothelial and mesenchymal cells is similar to what has been reported for Runx1 expressing cells in the AGM and suggests that Runx1 may also be involved in the generation of HSCs in the placenta (North et al., 1999; North et al., 2002).

TABLE 1
Adult repopulating HSC activity increases in the
placenta at different developmental stages.

| | | Number of mice | |
|---|---|---|---|
| | Placenta | repopulated/total | Range of |
| Stage | equivalent | transplanted | repopulation |
| E9 | 2-4 | 0/3 | 0 |
| E10 | | | |
| <35 SP | 3 | 0/2 | 0 |
| >35 SP | 1-3 | 0/4 | 0 |
| E11 | 0.5-3 | 1/5 | 100% |
| E12 | 1 | 3/3 | 15-62% |
| | 0.3 | 3/4 | 33-61% |
| | 0.1 | 3/6 | 48-67% |
| E12 GFP⁺ | 1 | 3/6 | 28-100% |
| | 0.25 | 4/9 | 32-100% |
| E12 GFP⁻ | 1 | 0/6 | 0 |
| | 0.25 | 0/8 | 0 |

E = embryonic day; SP = somite pairs.

TABLE 2
Multicolor flow cytometric analysis of E12 placental cells.

| | | | absolute |
|---|---|---|---|
| | % of total | % of GFP⁺ | number/placenta |
| GFP⁺ | 2.54 ± 0.47 | | 15216 ± 2809 |
| GFP⁺ ckit⁺ CD34⁺ | | 54.70 ± 11.27 | 7958 ± 1429 |
| GFP⁺ ckit⁺ CD31⁺ | | 55.26 ± 16.74 | 8654 ± 3237 |
| GFP⁺ ckit⁺ CD41⁺ | | 32.62 ± 7.43 | 5044 ± 1508 |
| GFP⁺ ckit⁺ CD45⁺ | | 11.88 ± 2.32 | 1743 ± 424 |

TABLE 3
Frequencies and estimated numbers of HSCs per
AGM obtained after explant culture in absence
or presence of different doses of IL-3.

| Explant | | HSCs/ | Amplification |
|---|---|---|---|
| culture + IL-3 | f_{HSCs} | AGM | factor |
| 0 | 1/165709 | 1.7 | — |
| 2 ng/ml | 1/49713 | 5.9 | 3.5 |
| 20 ng/ml | 1/19885 | 14.7 | 8.6 |
| 200 ng/ml | 1/4971 | 58.6 | 34.5 |

TABLE 4
Human placentas routinely contain higher percentages
of phenotypically-defined HSCs than cord blood.

| | | Percentage of cells | |
|---|---|---|---|
| | | CD34⁺ | CD34⁺CD38⁻⁻ |
| Placenta 1 | Cord Blood | 0.36 | 0.05 |
| | Placenta | nd | nd |
| Placenta 2 | Cord Blood | 0.41 | 0.03 |
| | Placenta | 2.39 ± 1.01 | 0.27 ± 0.11 |
| Placenta 3 | Cord Blood | 0.13 | 0.01 |
| | Placenta - villi | 0.26 ± 0.11 | 0.11 ± 0.04 |
| | Placenta - vascular | 1.18 | 0.26 |
| Placenta 4 | Cord Blood | nd | nd |
| | Placenta | 2.68 ± 0.59 | 0.52 ± 0.15 |
| Placenta 7 | Cord Blood | 0.37 | nd |
| | Placenta - villi | 4.70 | nd |
| | Placenta - vascular | 6.45 | nd |

FACS results of 5 individual placentas are presented here. The percentages of CD34⁺ and CD34⁺38⁻⁻ cells may vary between placentas due to differences in cell extraction procedures.

TABLE 5
CD34⁺ placenta cells are efficiently recovered after storage

| | number of cells | % CD34⁺ cells | % CD34⁺ cells |
|---|---|---|---|
| | per g tissue | pre-storage | post-storage |
| Cord Blood | nd | 0.36 | 0.76 |
| Placenta villi | 148,300 | 2.96 | 4.15 |
| Placenta total | 157,060 | 2.20 | 3.17 |

Human placenta cells were obtained and analysed by FACS for CD34 expression. Thereafter, cells were frozen in liquid nitrogen according to standard procedure. After storage for several weeks, cells were thawed and analysed by FACS for CD34 expression. Percentages of CD34⁺ cells in the placenta post-storage is increased as compared to pre-storage, indicating that freezing does not negatively affect human placenta CD34⁺ cells. Instead, freezing may lead to an enrichment of these cells.

### REFERENCES

Alvarez-Silva, M., Belo-Diabangouaya, P., Salaun, J., and Dieterlen-Lievre, F. (2003). Mouse placenta is a major hematopoietic organ. Development 130, 5437-5444.
Benito, A. I., Diaz, M. A., Gonzalez-Vicent, M., Sevilla, J., and Madero, L. (2004). Hematopoietic stem cell transplantation using umbilical cord blood progenitors: review of current clinical results. Bone Marrow Transplant 33, 675-690.
Bernex, F., De Sepulveda, P., Kress, C., Elbaz, C., Delouis, C., and Panthier, J. J. (1996). Spatial and temporal patterns of c-kit-expressing cells in WlacZ/+ and WlacZ/WlacZ mouse embryos. Development 122, 3023-3033.
Bonnet, D. (2002). Haematopoietic stem cells. J Pathol 197, 430-440.
Burgess, A. W., Wilson, E. M., and Metcalf, D. (1977). Stimulation by human placental conditioned medium of hemopoietic colony formation by human marrow cells. Blood 49, 573-583.
Cai, Z., de Bruijn, M., Ma, X., Dortland, B., Luteijn, T., Downing, R. J., and Dzierzak, E. (2000). Haploinsufficiency of AML1 affects the temporal and spatial generation of hematopoietic stem cells in the mouse embryo. Immunity 13, 423-431. Caprioli, A., Jaffredo, T., Gautier, R., Dubourg, C., and Dieterlen-Lievre, F. (1998). Blood-borne seeding by hematopoietic and endothelial precursors from the allantois. Proc Natl Acad Sci USA 95, 1641-1646.
Chen, C. Z., Li, L., Li, M., and Lodish, H. F. (2003). The endoglinpositive) sca-1(positive) rhodamine(low) phenotype defines a near-homogeneous population of long-term repopulating hematopoietic stem cells. Immunity 19, 525-533.
Cohen, Y., and Nagler, A. (2004). Umbilical cord blood transplantation—how, when and for whom? Blood Rev 18, 167-179.
Cross, J. C. (1998). Formation of the placenta and extraembryonic membranes. Ann N Y Acad Sci 857, 23-32.
Cross, J. C., Baczyk, D., Dobric, N., Hemberger, M., Hughes, M., Simmons, D. G., Yamamoto, H., and Kingdom, J. C. (2003a). Genes, development and evolution of the placenta. Placenta 24, 123-130.
Cross, J. C., Simmons, D. G., and Watson, E. D. (2003b). Chorioallantoic molphogenesis and formation of the placental villous tree. Ann N Y Acad Sci 995, 84-93.
de Bruijn, M. F., Ma, X., Robin, C., Ottersbach, K., Sanchez, M. J., and Dzierzak, E. (2002). Hematopoietic stein cells localize to the endothelial cell layer in the midgestation mouse aorta. Immunity 16, 673-683.
de Bruijn, M. F., Speck, N. A., Peeters, M. C., and Dzierzak, E. (2000). Definitive hematopoietic stem cells first develop within the major arterial regions of the mouse embryo. Embo J 19, 2465-2474.
de Vries, E. G., Vellenga, E., Kluin-Nelemans, J. C., and Mulder, N. H. (2004). The happy destiny of frozen haematopoietic stem cells: from immature stem cells to mature applications. Eur J Cancer 40, 1987-1992.
Dieterlen-Lievre, F. (1998). Hematopoiesis: progenitors and their genetic program. Curr Biol 8, R727-730.
Downs, K. M., Gifford, S., Blahnik, M., and Gardner, R. L. (1998). Vascularization in the murine allantois occurs by vasculogenesis without accompanying erythropoiesis. Development 125, 4507-4520.
Downs, K. M., and Harmann, C. (1997). Developmental potency of the murine allantois. Development 124, 2769-2780.
Fukuchi, Y., Nakajima, H., Sugiyama, D., Hirose, I., Kitamura, T., and Tsuji, K. (2004). Human placenta-derived cells have mesenchymal stem/progenitor cell potential. Stem Cells 22, 649-658.
Garcia-Porrero, J. A., Manaia, A., Jimeno, J., Lasky, L. L., Dieterlen-Lievre, F., and Godin, I. E. (1998). Antigenic profiles of endothelial and hemopoietic lineages in murine intraembryonic hemogenic sites. Dev Comp Immunol 22, 303-319.
Han, V. K., and Carter, A. M. (2001). Control of growth and development of the feto-placental unit. Curr Opin Pharmacol 1, 632-640.
Ikuta, K., and Weissman, I. L. (1992). Evidence that hematopoietic stem cells express mouse c-kit but do not depend on steel factor for their generation. Proc Natl Acad Sci USA 89, 1502-1506.
Ito, T., Tajima, F., and Ogawa, M. (2000). Developmental changes of CD34 expression by murine hematopoietic stem cells. Exp Hematol 28, 1269-1273.
Jaffredo, T., Gautier, R., Eichmann, A., and Dieterlen-Lievre, F. (1998). Intraaortic hemopoietic cells are derived from endothelial cells during ontogeny. Development 125.
Johnson, R. S., Spiegelman, B. M., and Papaioannou, V. (1992). Pleiotropic effects of a null mutation in the c-fos proto-oncogene. Cell 71, 577-586.
Keller; G., Lacaud, G., and Robertson, S. (1999). Development of the hematopoietic system in the mouse. Exp Hematol 27, 777-787.
Kliman, H. J., Nestler, J. E., Sermasi, E., Sanger, J. M., and Strauss, J. F. (1986). Pruification, characterization and in vitro differentiation of cytotrophoblasts from human term placentae. Endocrinology 118, 1567, 1582.
Kumaravelu, P., Hook, L., Morrison, A. M., Ure, J., Zhao, S., Zuyev, S., Ansell, J., and Medvinsky, A. (2002). Quantitative developmental anatomy of definitive haematopoietic stem cells/long-term repopulating units (HSC/RUs): role of the aorta-gonad-mesonephros (AGM) region and the yolk sac in colonisation of the mouse embryonic liver. Development 129, 4891-4899.
Labastie, M. C., Cortes, F., Romeo, P. H., Dulac, C., and Peault, B. (1998). Molecular identity of hematopoietic precursor cells emerging in the human embryo. Blood 92, 3624-3635.
Lansdorp, P. M., Dragowska, W., and Mayani, H. (1993). Ontogeny-related changes in proliferative potential of human hematopoietic cells. J Exp Med 178, 787-791.
Ling, K. W., Ottersbach, K., van Hamburg, J. P., Oziemlak, A., Tsai, F. Y., Orkin, S. H., Ploemacher, R., Hendriks, R. W., and Dzierzak, E. (2004). GATA-2 plays two functionally distinct roles during the ontogeny of hematopoietic stem cells. J Exp Med 200, 871-882.
Ma, X., Robin, C., Ottersbach, K., and Dzierzak, E. (2002). The Ly-6A (Sca-1) GFP Transgene is Expressed in all Adult Mouse Hematopoietic Stem Cells. Stem Cells 20, 514-521.
Ma, G. T., Roth, M. E., Groskopf, J. C., Tsai, F. Y., Orkin, S. H., Grosveld, F., Engel, J. D., and Linzer, D. I. (1997). GATA-2 and GATA-3 regulate trophoblast-specific gene expression in vivo. Development 124, 907-914.
Marshall, C. J., Moore, R. L., Thorogood, P., Brickell, P. M., Kinnon, C., and Thrasher, A. J. (1999). Detailed characterization of the human aorta-gonad-mesonephros region reveals morphological polarity resembling a hematopoietic stromal layer. Dev Dyn 215, 139-147.
Marshall, C. J., and Thrasher, A. J. (2001). The embryonic origins of human haematopoiesis. Br J Haematol 112, 838-850.
Medvinsky, A. and Dzierzak, E. (1996) Definitive hematopoiesis is autonomously initiated by the AGM region. Cell 86, 897-906.
Mikkola, H. K. A., Gekas, C., Dieterlen-Lievre, F., and Orlcin, S. H. (2004). Placenta is a niche for hematopoietic stem cells. Blood 104, 730a.
Minegishi, N., Ohta, J., Yamagiwa, H., Suzuki, N., Kawauchi, S., Zhou, Y., Talcahashi, S., Hayashi, N., Engel, J. D., and Yamamoto, M. (1999). The mouse GATA-2 gene is expressed in the para-aortic splanchnopleura and aorta-gonads and mesonephros region. Blood 93, 4196-4207.
Morrison, S. J., Wandycz, A. M., Akashi, K., Globerson, A., and Weissman, I. L. (1996). The aging of hematopoietic stem cells. Nat Med 2, 1011-1016.
Muller, A. M., Medvinsky, A., Strouboulis, J., Grosveld, F., and Dzierzak, E. (1994). Development of hematopoietic stem cell activity in the mouse embryo. Immunity 1, 291-301.
Muller, R., Verma, I. M., and Adamson, B. D. (1983). Expression of c-onc genes: c-fos transcripts accumulate to high levels during development of mouse placenta, yolk sac and amnion. Embo J 2, 679-684.
Ng, Y. K., George, K. M., Engel, J. D., and Linzer, D. I. (1994). GATA factor activity is required for the trophoblast-specific transcriptional regulation of the mouse placental lactogen I gene. Development 120, 3257-3266.
Nishikawa, S. I. (2001). A complex linkage in the developmental pathway of endothelial and hematopoietic cells. Curr Opin Cell Biol 13, 673-678.
North, T., Gu, T.-L., Stacy, T., Wang, Q., Howard, L., Binder, M., Marin-Padilla, M., and Speck, N. (1999). Cbfα is required for the formation of intraaortic hematopoietic clusters. Development 126, 2563-2575.
North, T. E., de Bruijn, M. F., Stacy, T., Talebian, L., Lind, E., Robin, C., Binder, M., Dzierzak, E., and Speck, N. A. (2002). Runx1 expression marks long-term repopulating hematopoietic stein cells in the midgestation mouse embryo. Immunity 16, 661-672.
Oberlin, E., Tavian, M., Blazsek, B., and Peault, B. (2002). Blood-forming potential of vascular endothelium in the human embryo. Development 129, 4147-4157.
Ogawa, M. (2002). Changing phenotypes of hematopoietic stem cells. Exp Hematol 30, 3-6.
Okuda, T., van Deursen, J., Hiebert, S. W., Grosveld, G., and Downing, J. R. (1996). AML1, the target of multiple chromosomal translocations in human leukemia, is essential for normal fetal liver hematopoiesis. Cell 84, 321-330.
Osawa, M., Hanada, K., Hamada, H., and Nakauchi, H. (1996). Long-term lymphohematopoietic reconstitution by a single CD34-low/negative hematopoietic stem cell. Science 273, 242-245.
Pamphilon, D. (2004). Stem-cell harvesting and manipulation. Vox Sang 87 Suppl1, 20-25.
Pandolfi, P. P., Roth, M. E., Karis, A., Leonard, M. W., Dzierzak, E., Grosveld, F. G., Engel, J. D., and Lindenbaum, M. H. (1995). Targeted disruption of the GATA3 gene causes severe abnormalities in the nervous system and in fetal liver haematopoiesis. Nat Genet 11, 40-44.
Peault, B. (1996). Hematopoietic stem cell emergence in embryonic life: developmental hematology revisited. J Hematother 5, 369-378.
Peterson, D. A. (2004). Umbilical cord blood cells and brain stroke injury: bringing in fresh blood to address an old problem. J Clin Invest 114, 312-314.
Robin, C., Ottersbach, K. and Dzierzak, E. (2004) Hematopoietic stem cells during embryonic development. In: Pluripotential Hematopoietic Stem Cell (PHSC). Ed. J. R. Keller. Landes Bioscience.
Rocha, V., Sanz, G., and Gluckman, E. (2004). Umbilical cord blood transplantation. Curr Opin Hematol 11, 375-385.
Romanov, Y. A., Svintsitskaya, V. A., and Smimov, V. N. (2003). Searching for alternative sources of postnatal human mesenchymal stem cells: candidate MSC-like cells from umbilical cord. Stem Cells 21, 105-110.
Rossant, J., and Cross, J. C. (2001). Placental development: lessons from mouse mutants. Nat Rev Genet 2, 538-548.
Sanchez, M. J., Holmes, A., Miles, C., and Dzierzak, E. (1996). Characterization of the first definitive hematopoietic stem cells in the AGM and liver of the mouse embryo. Immunity 5, 513-525.
Shivdasani, R. A., Mayer, E. L., and Orldn, S. H. (1995). Absence of blood formation in mice lacking the T-cell leukaemia oncoprotein tal-1/SCL. Nature 373, 432-434.
Spangrude, G. J., Heimfeld, S., and Weissman, I. L. (1988). Purification and characterization of mouse hematopoietic stem cells. Science 241, 58-62.
Tavian, M., Coulombel, L., Luton, D., Clemente, H. S., Dieterlen-Lievre, F., and Peault, B. (1996). Aorta-associated CD34+ hematopoietic cells in the early human embryo. Blood 87, 67-72.
Tavian, M., Hallais, M. F., and B, P. a. (1999). Emergence of intraembryonic hematopoietic precursors in the pre-liver human embryo. Development 126, 793-803.
Tavian, M., Robin, C., Coulombel, L., and Peault, B. (2001). The human embryo, but not its yolk sac, generates lympho-myeloid stem cells: mapping multipotent hematopoietic cell fate in intraembryonic mesoderm. Immunity 15, 487-495.
Tsai, F. Y., Keller, G., Kuo, F. C., Weiss, M., Chen, J., Rosenblatt, M., Alt, F. W., and Orkin, S. H. (1994). An early haematopoietic defect in mice lacking the transcription factor GATA-2. Nature 371, 221-226.
van Doorninck, J. H., van Der Wees, J., Karis, A., Goedknegt, E., Engel, J. D., Coesmans, M., Rutteman, M., Grosveld, F., and De Zeeuw, C. I. (1999). GATA-3 is involved in the development of serotonergic neurons in the caudal raphe nuclei. J Neurosci 19, RC12.
Wang, Q., Stacy, T., Binder, M., Marin-Padilla, M., Sharpe, A. H., and Speck, N. A. (1996). Disruption of the Cbfa2 gene causes necrosis and hemorrhaging in the central nervous system and blocks definitive hematopoiesis. Proc Natl Acad Sci USA 93, 3444-3449.
Wulf, G. G., Viereck, V., Hemmerlein, B., Haase, D., Vehmeyer, K., Pulcrop, T., Glass, B., Emons, G., and Trumper, L. (2004). Mesengenic progenitor cells derived from human placenta. Tissue Eng 10, 1136-1147.
Zhang, Y., Li, C., Jianig, X., Zhang, S., Wu, Y., Liu, B., Tang, P., and Mao, N. (2004). Human placenta-derived mesenchymal progenitor cells support culture expansion of long-term culture-initiating cells from cord blood CD34+ cells. Exp Hematol 32, 657-664.
Zhou, Y., Lim, K. C., Onodera, K., Talcahashi, S., Ohta, J., Minegishi, N., Tsai, F. Y., Orkin, S. H., Yamamoto, M., and Engel, J. D. (1998). Rescue of the embryonic lethal hematopoietic defect reveals a critical role for GATA-2 in urogenital development. Embo J 17, 6689-6700.
Zhu, J., and Emerson, S. G. (2002). Hematopoietic cytokines, transcription factors and lineage commitment. Oncogene 21, 3295-3313.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology and biotechnology or related fields are intended to be within the scope of the following claims.

1. A method for obtaining a population of human haemopoietic stem-cells (HSCs) which method comprises extracting said cells from a placenta of a human individual post-parturn.

2. The method according to claim 1 wherein extracting the HSCs from said placenta comprises the process of sorting the cells using a HSC cell surface marker.

3. The method according to claim 1 or claim 2 wherein the cell surface marker is one or more markers selected from the group consisting of: Sca1 and CD34 and CD38 and Thy1.

4. The method according to claim 2 wherein the sorting method comprises the use of flow cytometry.

5. A population of haemopoietic stem cells (HSCs) isolated from the placenta of a human individual post-partum.

6. The population of haemopoietic stem cells (HSCs) according to claim 5 which have potent multi-lineage cell repopulation activity.

7. The population of human haernopoietic stem cells (HSCs) according to claim 6 which have a more potent multi-lineage cell repopulation activity than a similar number of HSCs extracted from one or more sources of HSCs selected from the group consisting of: cord blood, bone marrow and peripheral blood.

8. A population of human haernopoietic stem cells (HSCs) according to any of claim 5, 6 and 7, wherein the number of HSCs within that population has been increased by (i) treatment of said population with one or more growth factors selected from the group consisting of: IL-3, EL-6, Tpo, OSM, SCF, GM-CSF, MIP1γ, Wnt, BMP and NGFf3; and/or (ii) treating them with explant cultures derived from placenta; (iii) and/or treating said population with explant cultures derived from AGM and/or reaggregation cultures and/or stromal cell cocultures.

9. A method for providing a population of human HSCs comprising the steps of: (a) Extracting a population of HSCs from human post-partum placenta, and (b) Treating said population of cells with one or more growth factors selected from the group consisting of: IL-3, IL-6, Tpo, OSM, SCF, GM-CSF, MIP1y, Wnt, BMP, NGF; treating them with explant cultures derived from placenta, and/or treating them with explant cultures derived from AGM and/or reaggregation cultures and/or stromal cell cocultures.

10. A method of populating a variety of non-hematopoietic tissues of an individual with HSCs isolated from the placenta of a human indificual post-partum in and/or the progeny of those cells obtained after ex vivo culture.

11. The method of claim 10, wherein those cells possess one or more of the features of claims 5 to 8.

12. The use of one or more human haemopoietic stem cells (HSCs) according to any of claims 5 to 8 in populating a human individual with haemopoietic stem cells.

13. A population of haemopoietic stem cells isolated from the placenta of a human individual post-partum that maintain their viability and function after storage.
